(19) United States
(12) Patent Application Publication
(10) Pub. No.: US 2023/0363679 A1
(43) Pub. Date: Nov. 16, 2023

(54) SYSTEMS AND METHODS FOR ASSISTING WITH STROKE AND OTHER NEUROLOGICAL CONDITION DIAGNOSIS USING MULTIMODAL DEEP LEARNING

(71) Applicants: THE PENN STATE RESEARCH FOUNDATION, UNIVERSITY PARK, PA (US); THE METHODIST HOSPITAL, Houston, TX (US)

(72) Inventors: James Z. Wang, State College, PA (US); Mingli Yu, State College, PA (US); Tongan Cai, State College, PA (US); Xiaolei Huang, State College, PA (US); Kelvin Wong, Houston, TX (US); John Volpi, University Park, PA (US); Stephen T.C. Wong, University Park, PA (US)

(73) Assignees: THE PENN STATE RESEARCH FOUNDATION, UNIVERSITY PARK, PA (US); THE METHODIST HOSPITAL, Houston, TX (US)

(21) Appl. No.: 18/025,974

(22) PCT Filed: Sep. 17, 2021
(86) PCT No.: PCT/US2021/050869

§ 371 Date: Mar. 13, 2023

Related U.S. Application Data
(60) Provisional application No. 63/079,722, filed on Sep. 17, 2020.

(51) Int. Cl. A61B 5/16; A61B 5/00; A61B 5/1171

(52) U.S. Cl. CPC A61B 5/165; A61B 5/7267; A61B 5/1176; A61B 5/4803; A61B 5/0077

(57) ABSTRACT

A system includes a mobile device for capturing raw video of a subject, a preprocessing system communicatively coupled to the mobile device for splitting the raw video into an image stream and an audio stream, an image processing system communicatively coupled to the preprocessing system for processing the image stream into a spatiotemporal facial frame sequence proposal, an audio processing system for processing the audio stream into a preprocessed audio component, one or more machine learning devices that analyze the facial frame sequence proposal and the preprocessed audio component according to a trained model to determine whether the subject is exhibiting signs of a neurological condition, and a user device for receiving data corresponding to a confirmed indication of neurological condition from the one or more machine learning devices and providing the confirmed indication of neurological condition to the subject and/or a clinician via a user interface.

### CROSS-REFERENCE TO RELATED APPLICATION

The present application claims the priority benefit of U.S. Provisional Patent Application Ser. No. 63/079,722, entitled “SYSTEMS AND METHODS FOR ASSISTING WITH STROKE DIAGNOSIS USING MULTIMODAL DEEP LEARNING” and filed Sep. 17, 2020, which is incorporated by reference herein in its entirety.

### BACKGROUND

### Field

The present specification generally relates to assessing neurological conditions such as stroke and, more particularly, to systems, methods, and storage media for using machine learning to analyze the presence of a neurological condition.

### Technical Background

Stroke is a common and potentially fatal vascular disease. Stroke is the second leading cause of death and the third leading cause of disability. A common form of stroke is acute ischemic stroke, where parts of the brain tissue suffer from restrictions in blood supply to tissues, and the shortage of oxygen needed for cellular metabolism quickly causes long-lasting damage to the areas of brain cells. The sooner a diagnosis is made, the earlier the treatment can begin and the more likely a subject will have a good outcome with less disability and lower likelihood of recurrent vascular disease.

There is no rapid assessment approach for stroke. One test for stroke is a diffusion-weighted MRI scan that detects brain ischemia, but is usually not accessible in an emergency room setting. Two commonly adopted clinical tests for stroke in the emergency room include the Cincinnati Pre-hospital Stroke Scale (CPSS) and the Face Arm Speech Test (FAST). Both tests assess the presence of any unilateral facial droop, arm drift, and speech disorder. The subject is requested to repeat a specific sentence (CPSS) or have a conversation with the doctor (FAST), and abnormality arises when the subject slurs, fails to organize his speech, or is unable to speak. However, the scarcity of neurologists prevents such tests to be effectively conducted in all stroke emergency situations.

### SUMMARY

One aspect of the present disclosure relates to method that includes receiving, by a processing device, raw video of a subject presented for potential neurological condition, splitting, by the processing device, the raw video into an image stream and an audio stream, preprocessing the image stream into a spatiotemporal facial frame sequence proposal, preprocessing the audio stream into a preprocessed audio component, transmitting the facial frame sequence proposal and preprocessed audio component to a machine learning device that analyzes the facial frame sequence proposal and the preprocessed audio component according to a trained model to determine whether the subject is exhibiting signs of a neurological condition, receiving, from the machine learning device, data corresponding to a confirmed indication of neurological condition, and providing the confirmed indication of neurological condition to the subject and/or a clinician via a user interface.

Another aspect of the present disclosure relates to a system that includes at least one processing device and a non-transitory, processor readable storage medium. The non-transitory, processor readable storage medium includes programming instructions thereon that, when executed, cause the at least one processing device to receive raw video of a subject presented for potential neurological condition, split the raw video into an image stream and an audio stream, preprocess the image stream into a spatiotemporal facial frame sequence proposal, preprocess the audio stream into a preprocessed audio component, transmit the facial frame sequence proposal and preprocessed audio component to a machine learning device that analyzes the facial frame sequence proposal and the preprocessed audio component according to a trained model to determine whether the subject is exhibiting signs of a neurological condition, receive, from the machine learning device, data corresponding to a confirmed indication of neurological condition, and provide the confirmed indication of neurological condition to the subject and/or a clinician via a user interface.

Yet another aspect of the present disclosure relates to non-transitory storage medium that includes programming instructions thereon for causing at least one processing device to receive raw video of a subject presented for potential neurological condition, split the raw video into an image stream and an audio stream, preprocess the image stream into a spatiotemporal facial frame sequence proposal, preprocess the audio stream into a preprocessed audio component, transmit the facial frame sequence proposal and preprocessed audio component to a machine learning device that analyzes the facial frame sequence proposal and the preprocessed audio component according to a trained model to determine whether the subject is exhibiting signs of a neurological condition, receive, from the machine learning device, data corresponding to a confirmed indication of neurological condition, and provide the confirmed indication of neurological condition to the subject and/or a clinician via a user interface.

Yet another aspect of the present disclosure relates to a system that includes a mobile device for capturing raw video of a subject presented for potential neurological condition, a preprocessing system communicatively coupled to the mobile device for splitting the raw video into an image stream and an audio stream, an image processing system communicatively coupled to the preprocessing system for processing the image stream into a spatiotemporal facial frame sequence proposal, an audio processing system for processing the audio stream into a preprocessed audio component, one or more machine learning devices that analyze the facial frame sequence proposal and the preprocessed audio component according to a trained model to determine whether the subject is exhibiting signs of a neurological condition, and a user device for receiving data corresponding to a confirmed indication of neurological condition from the one or more machine learning devices and providing the confirmed indication of neurological condition to the subject and/or a clinician via a user interface.

These and other features, and characteristics of the present technology, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention. As used in the specification and in the claims, the singular form of ‘a’, ‘an’, and ‘the’ include plural referents unless the context clearly dictates otherwise.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, wherein like structure is indicated with like reference numerals and in which:

FIG. 1 schematically depicts an illustrative network of devices and systems for assessing a neurological condition according to one or more embodiments shown and described herein;

FIG. 2A depicts illustrative internal components of a mobile device according to one or more embodiments shown and described herein;

FIG. 2B depicts a block diagram of illustrative logic modules of a memory component of a mobile device according to one or more embodiments shown and described herein;

FIG. 3A depicts illustrative internal components of one or more machine learning devices according to one or more embodiments shown and described herein;

FIG. 3B depicts a block diagram of illustrative logic modules of a memory component of one or more machine learning devices according to one or more embodiments shown and described herein;

FIG. 4A depicts an illustrative printed image that is used for assessment of a subject according to one or more embodiments shown and described herein;

FIG. 4B schematically depicts a subject holding a mobile device for the purposes of assessing a neurological condition according to one or more embodiments shown and described herein;

FIG. 5A schematically depicts a first portion of a block diagram of an illustrative training framework according to one or more embodiments shown and described herein;

FIG. 5B schematically depicts a second portion of a block diagram of an illustrative training framework according to one or more embodiments shown and described herein;

FIG. 5C schematically depicts a third portion of a block diagram of an illustrative training framework according to one or more embodiments shown and described herein;

FIG. 6A depicts a flow diagram of an illustrative image data preprocessing process according to one or more embodiments shown and described herein;

FIG. 6B depicts a flow diagram relating to speech spectrum analysis according to one or more embodiments shown and described herein;

FIG. 7 depicts a flow diagram of an illustrative process of extracting features from images and a spectrogram according to one or more embodiments shown and described herein; and

FIG. 8 depicts an ROC curve that shows the model performance of an illustrative model, other baselines, and the performance of the clinicians in the ER according to one or more embodiments shown and described herein.

### DETAILED DESCRIPTION

The present disclosure relates generally to training and using a deep learning framework that can later be used for the purposes of clinical assessment of a neurological condition, such as stroke or the like in an emergency room or other clinical care type setting where a relatively quick assessment is desirable. The deep learning framework is used to achieve computer-aided stroke presence assessment by recognizing the patterns of facial motion incoordination and speech inability for subjects with suspicion of stroke or other neurological condition in an acute setting. The deep learning framework takes two modalities of data: video data for local facial paralysis detection and audio data for global speech and cognitive disorder analysis. The framework further leverages a multi-modal lateral fusion to combine the low-level and high-level features and provides mutual regularization for joint training. A novel adversarial training loss is also introduced to obtain identity-independent and stroke-discriminative features. Experiments on a video-audio dataset used by the framework with actual subjects show that the approach outperforms several state-of-the-art models and achieves better diagnostic performance than ER doctors, attaining a 6.60% higher sensitivity rate and maintaining 4.62% higher accuracy when specificity is aligned. Meanwhile, each assessment can be completed in less than six minutes on a personal computer, demonstrating the great potential of clinical implementation.

The algorithm and framework discussed herein aims to let the deep learning model to determine the existence of a stroke with both audio and video data. While there are some clinical indicators of stroke (i.e., facial drop, dysphasia) that has been referred to by doctors, the framework of the present disclosure seeks a more data-driven way that doesn't require an assumption of knowledge. The network relies on the “deep features” extracted to perform the classification, but the patterns in the features may not be intuitive since audio features to video features are concatenated at multiple stages.

The deep learning framework generally detects “deep features” that are mathematically represented in the trained models, but the models are not meant for humans to interpret generally. That's why the present disclosure does not specifically relate to stroke features that are evident when a human views a potential stroke victim. The deep learning approach is quite different from conventional machine learning approaches where models generated can sometimes be interpreted easily by humans.

Stroke is a common cerebrovascular disease that can cause lasting brain damage, long-term disability, or even death. It is the second leading cause of death and the third leading cause of disability worldwide. Someone in the United States has a stroke every forty seconds and someone dies of a stroke every four minutes. In acute ischemic stroke where brain tissue lacks blood supply, the shortage of oxygen needed for cellular metabolism quickly causes long-lasting tissue damage. If identified and treated in time, an acute ischemic stroke subject will have a greater chance of survival and subsequently a better quality of life with a lower chance of recurrent vascular disease.

However, delays are inevitable during the presentation, evaluation, diagnosis, and treatment of stroke. There is no reliable and rapid assessment approach for stroke. Currently, one test for stroke is advanced neuro-imaging including diffusion-weighted MRI scan (DWI) that detects brain infarct with high sensitivity and specificity. Although accurate, DWI is usually not accessible in the emergency room (ER) due to limited equipment availability, turn around time for subject transport and scanning, and high operating cost. Therefore, in the typical ER scenario, clinicians commonly adopt the following three tests: the Cincinnati Pre-hospital Stroke Scale (CPSS), the Face Arm Speech Test (FAST), and the National Institutes of Health Stroke Scale (NIHSS). All these methods assess the presence of any unilateral facial droop, arm drift, and speech disorder. The subject is requested to repeat a specific sentence (CPSS) or have a conversation with the doctor (FAST), and abnormalities arise when the subject slurs, fails to organize his speech, or is unable to speak. For NIHSS, face and limb palsy conditions are also evaluated. However, the scarcity of neurologists and annual certification requirements for NIHSS makes such tests difficult to be timely and effectively conducted in all stroke emergencies. The evaluation may also fail to detect stroke cases where only very subtle facial motion deficits exist—that clinicians are unable to observe.

Some researchers are now focusing on alternative contactless, efficient, and economic ways for the analysis of various neurological conditions. One of the most popular domains is the detection of facial paralysis with computer vision by allowing machines to detect the anomalies in the subjects' faces. However, the majority of work neglects the readily available and indicative speech audio features, which can be an important source of information in stroke diagnosis. Also, current methods ignore the spatiotemporal continuity of facial motions and fail to tackle the problem of static/natural asymmetry. Common video classification frameworks like I3D and SlowFast also fail to serve the stroke pattern recognition purpose due to the lack of training data and quick overfitting as “subject-remembering” effect.

In addition, few datasets of high quality have been constructed in stroke diagnosis domain. The current clinical datasets are small (with hundreds of images or dozens of videos) and unable to comprehensively represent the diversity in stroke subjects in terms of gender, race/ethnicity, and age. Also, the datasets either evaluate between normal subjects versus those with clear signs of a stroke or deal with full synthetic data (e.g., healthy people that pretend to have palsy facial patterns). Some other datasets establish experimental settings with hard constraints on the subject's head. All of these datasets hinder their clinical implementation for ER screening or subject self-assessment.

In the present application, a novel deep learning framework is described to accurately and efficiently analyze the presence of stroke in subjects with suspicion of a stroke. The problem is formulated as a binary classification task (e.g., stroke vs. non-stroke). Instead of taking a single-modality input, the core network in the deep learning framework described herein consists of two temporal-aware branches, the video branch for local facial motion analysis and the audio branch for global vocal speech analysis, to collaboratively detect the presence of stroke patterns. A novel lateral connection scheme between these two branches is introduced to combine the low-level and high-level features and provide mutual regularization for joint training. To mitigate the “subject-remembering” effect, the deep learning framework described herein also makes use of adversarial learning to learn subject-independent and stroke-discriminative features for the network.

To evaluate the deep learning framework described herein, a stroke subject video/audio dataset was constructed. The dataset records the facial motions of the subjects during their process of performing a set of vocal speech tests when they visit a care site such as the ER, an urgent care center, or the like. The recruited participants are all showing some level of neurological conditions with a high risk of stroke when visiting a point of care, which is closer to real-life scenarios and much more challenging than distinguishing stroke subjects from healthy people or student training videos. The dataset includes diverse subjects of different genders, races/ethnicity, ages, and at different levels of stroke conditions; the subjects are free of motion constraints and are in arbitrary body positions, illumination conditions, and background scenarios, which can be regarded as “in-the-wild.” Experiments on the dataset show that the proposed deep learning framework can achieve high performance and even outperform trained clinicians for stroke diagnosis while maintaining a manageable computation workload.

As described in greater detail herein, the present disclosure describes construction of a real clinical subject facial video and vocal audio dataset for stroke screening with diverse participants. The videos are collected “in-the-wild,” with unconstrained subject body positions, environment illumination conditions, and background scenarios. Next, the deep learning multimodal framework highlights its video-audio multi-level feature fusion scheme that combines global context to local representation, the adversarial training that extracts identity-independent stroke features, and the spatiotemporal proposal mechanism for frontal human facial motion sequences. The proposed multi-modal method achieves high diagnostic performance and efficiency on the dataset and outperforms the clinicians, demonstrating its high clinical value to be deployed for real life use

The contributions of present disclosure are summarized in three aspects:

The present disclosure analyzes the presence of stroke among actual ER subjects with suspicion of stroke using computational facial motion analysis, and adopts a natural language processing (NLP) method for the speech ability test on at-risk stroke subjects.

A multi-modal fusion of video and audio deep learning models is introduced with 93.12% sensitivity and 79.27% accuracy in correctly identifying subjects with stroke, which is comparable to the clinical impression given by ER physicians. The framework can be deployed on a mobile platform to enable self-assessment for subjects right after symptoms emerge.

The proposed temporal proposal of human facial videos can be adopted in general facial expression recognition tasks. The proposed multi-modal method can potentially be extended to other clinical tests, especially for neurological disorders that result in muscular motion abnormalities, expressive disorder, and cognitive impairments.

It should be appreciated that while the present disclosure relates predominately to stroke diagnosis and/or assessment, the present disclosure is not limited to such. That is, the systems and methods described herein can be applied to a wide variety of other neurological conditions or suspected neurological conditions. More specifically, the framework used herein can be used to train the machine learning systems for stroke and/or other neurological conditions using the same or similar data inputs.

Furthermore, while the present disclosure relates to the assessment and/or diagnosis of stroke or other neurological conditions in an acute setting such as the ER by a doctor, the present disclosure is not limited to such. That is, due to the use of a mobile device, the assessment and/or diagnosis could be completed by any individual, including non-doctor medical personnel or the like, self completed, completed by a family member or caretaker, and/or the like. In addition, the assessment and/or diagnosis could be completed in non-acute medical settings, such as, for example, a doctor's office, a clinic, a facility such as a retirement home or nursing home, an individual's own home, or virtually any other location where a mobile device may be carried and/or used.

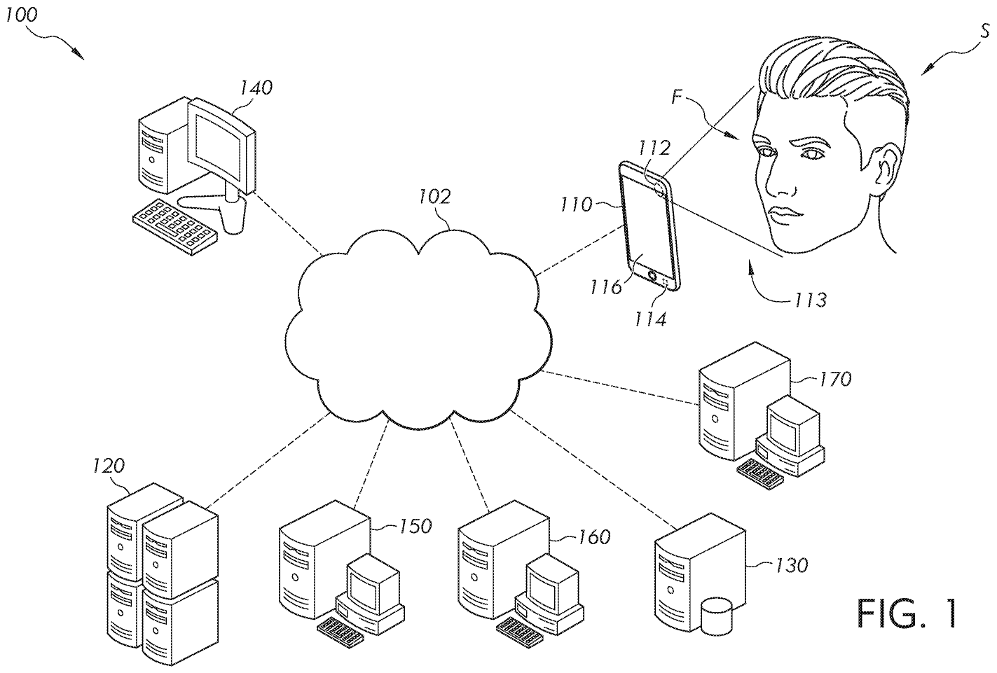

Referring now to the drawings, FIG. 1 depicts an illustrative system, generally designated 100, of networked devices and systems for assisting with stroke diagnosis using multimodal deep learning according to one or more embodiments shown and described herein. As illustrated in FIG. 1, the system 100 may include at network 102, such as, for example a wide area network (e.g., the internet), a local area network (LAN), a mobile communications network, a public service telephone network (PSTN) and/or other network and may be configured to electronically connect a mobile device 110, one or more machine learning devices 120, a database server 130, a user computing device 140, an audio processing system 150, an image processing system 160, and/or a preprocessing system 170.

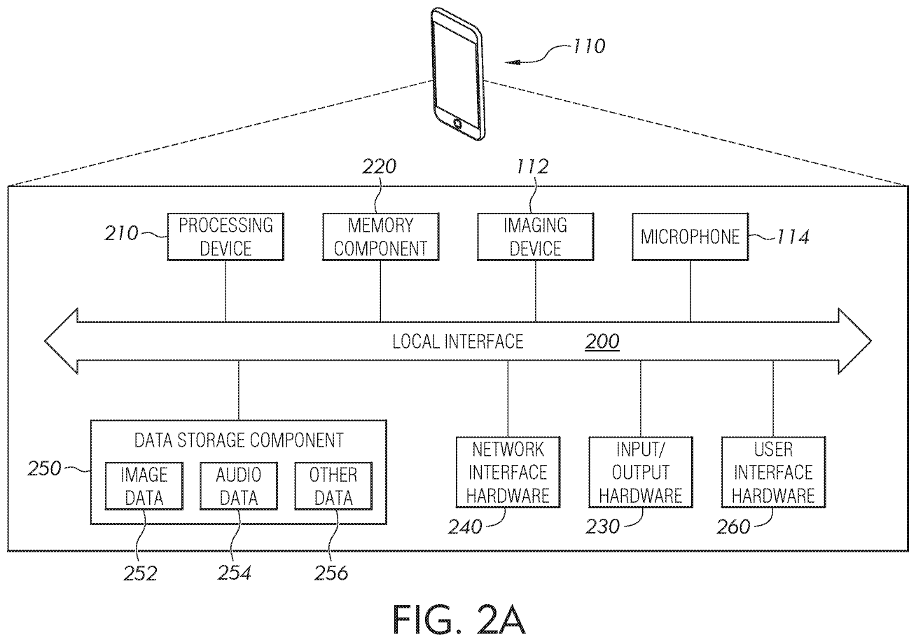

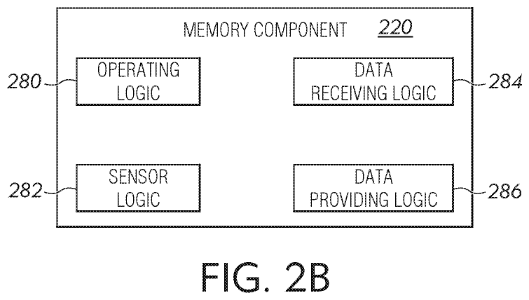

The mobile device 110 may generally include an imaging device 112 such as a camera having a field of view 113 that is capable of capturing images (e.g., pictures and/or an image portion of a video stream) of a face F of a subject S. The images may include raw video and/or a 3D depth data stream (e.g., captured by a depth sensor such as a LIDAR sensor or the like). The mobile device 110 may further include a microphone 114 or the like for capturing audio (e.g., an audio portion of a video stream). The mobile device also includes a display 116, which is used for displaying images and/or video to the user. It should be understood that while the mobile device 110 is depicted as a smartphone, this is a nonlimiting example. More specifically, in some embodiments, any type of computing device (e.g., mobile device, tablet computing device, personal computer, server, etc.) may be used. Further, in some embodiments, the imaging device 112 may be a front facing imaging device (e.g., a camera that faces the same direction as the display 116) so that the subject can view the display 116 and the imaging device 110 and the microphone 114 can capture video (e.g., audio and images) of the face F of the subject S in real time as the subject S is viewing the display 116. In other embodiments, the imaging device 112 may be a rear-facing camera on the smartphone and the display is a remote display or the like. Additional details regarding the mobile device 110 will be described herein with respect to FIGS. 2A-2B.

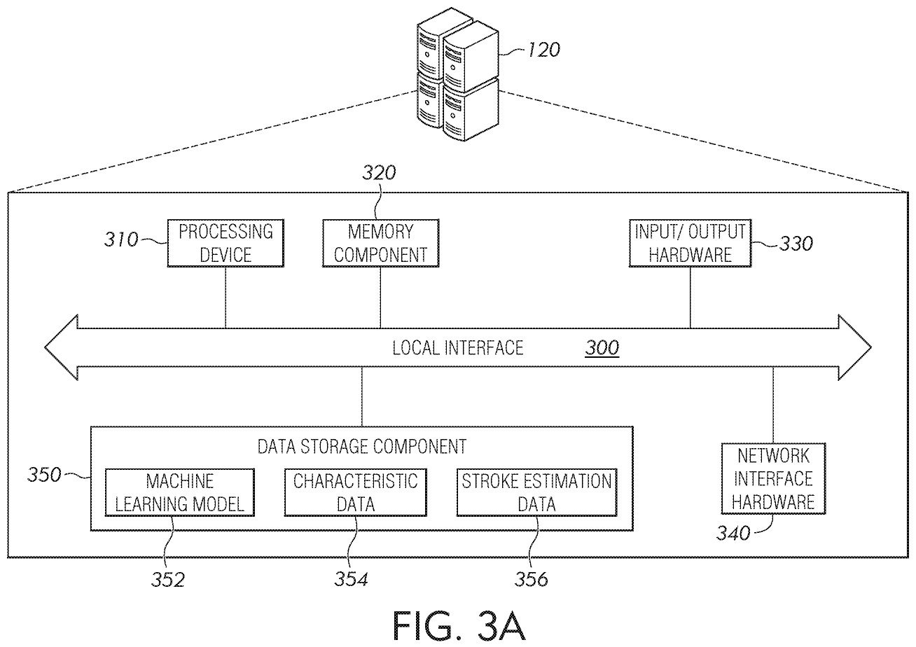

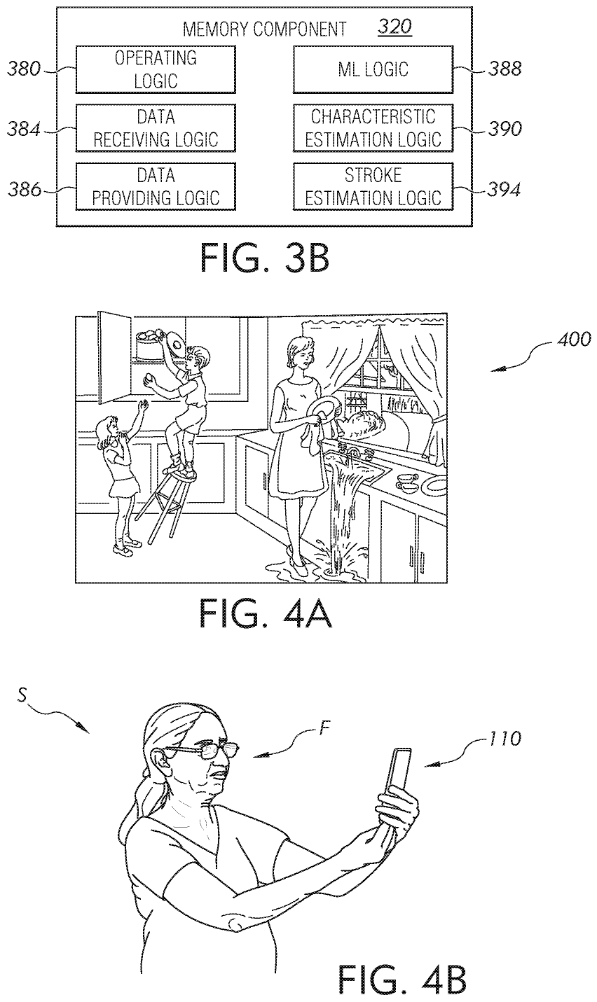

Still referring to FIG. 1, the one or more machine learning devices 120 are generally computing devices that store one or more machine learning algorithms thereon and are particularly configured to receive data from the mobile device 110 by way of the audio processing system 150, the image processing system 160, and/or the preprocessing system 170 and generate a model therefrom, the model being usable to assess stroke features, as described in greater detail herein. The machine learning algorithms utilized by the one or more machine learning devices 120 are not limited by the present disclosure, and may generally be any algorithm now known or later developed, particularly those that are specifically adapted for determining a likelihood that a stroke has occurred in a subject based on the audio and/or image data provided thereto, according to embodiments shown and described herein. That is, the machine learning algorithms may be supervised learning algorithms, unsupervised learning algorithms, semi-supervised learning algorithms, and reinforcement learning algorithms. Specific examples of machine learning algorithms may include, but are not limited to, nearest neighbor algorithms, naïve Bayes algorithms, decision tree algorithms, linear regression algorithms, supervised vector machines, neural networks, clustering algorithms, association rule learning algorithms, Q-learning algorithms, temporal difference algorithms, and deep adversarial networks. Other specific examples of machine learning algorithms used by the one or more machine learning devices 120 should generally be understood and are included within the scope of the present disclosure. Additional details regarding the one or more machine learning devices 120 will be described herein with respect to FIGS. 3A-3B.

Still referring to FIG. 1, the data server 130 may generally be a server computing device that is adapted to receive data from any of the components described herein and store the data. In addition, the data server 130 may further be adapted to provide the data to any one of the components described herein when requested. To complete such processes, the data server 130 may include various hardware components such as, for example, one or more processors, memory, data storage components (e.g., hard disc drives), communications hardware, and/or the like. For example, the data server 130 may include communications hardware that is usable to receive the data and/or transmit the data. In another example, the data server 130 may include data storage components for storing the data. The various hardware components of the data server 130 should generally be understood and is not described herein. In some embodiments, the data server 130 may be one or more cloud-based storage devices that are particularly adapted to receive, transmit, and store data.

The user computing device 140 may generally be a personal computing device, workstation, or the like that is particularly adapted to display and receive inputs to/from a user. The user computing device 140 may include hardware components that provide a graphical user interface that displays data, images, video and/or the like to the user, who may be, for example, a doctor, a nurse, a specialist, a caretaker, the subject, or the like. In some embodiments, the user computing device 140 may, among other things, perform administrative functions for the data server 130. That is, in the event that the data server 130 requires oversight, updating, or correction, the user computing device 140 may be configured to provide the desired oversight, updating, and/or correction. The user computing device 140 may also be utilized to perform other user-facing functions. To complete such processes and functions, the user computing device 140 may include various hardware components such as, for example, processors, memory, data storage components (e.g., hard disc drives), communications hardware, display interface hardware, user interface hardware, and/or the like.

The audio processing system 150 is generally a computing device, software, or the like that is particularly configured to process audio components of the video that is captured by the microphone 113 of the mobile computing device 110 and provide the processed audio components to various other components of the system 100, such as, for example, the machine learning devices 120. In some embodiments, such as the embodiment depicted in FIG. 1, the audio processing system 150 is a system that is separate and distinct from the other components of the system 100. In such embodiments, the audio processing system 150 may include various hardware components such as, for example, processors, memory, data storage components (e.g., hard disc drives), communications hardware, display interface hardware, user interface hardware, and/or the like. In some embodiments, the audio processing system 150 may be a service that is provided by a third-party provider. In other embodiments, the audio processing system 150 may be a hardware component or software that is integrated with one or more other components of the system, such as, for example, the mobile device 110 and/or the preprocessing system 170.

The image processing system 160 is generally a computing device, software, or the like that is particularly configured to process image components of the video that is captured by the imaging device 112 of the mobile computing device 110 and provide the processed image components to various other components of the system 100, such as, for example, the machine learning devices 120. In some embodiments, such as the embodiment depicted in FIG. 1, the image processing system 160 is a system that is separate and distinct from the other components of the system 100. In such embodiments, the image processing system 160 may include various hardware components such as, for example, processors, memory, data storage components (e.g., hard disc drives), communications hardware, display interface hardware, user interface hardware, and/or the like. In some embodiments, the image processing system 160 may be a service that is provided by a third-party provider. In other embodiments, the image processing system 160 may be a hardware component or software that is integrated with one or more other components of the system, such as, for example, the mobile device 110 and/or the preprocessing system 170.

The preprocessing system 170 is generally a computing device, software, or the like that is particularly configured to process the video obtained by the mobile computing device 110 (e.g., the image stream that is captured by the imaging device 112 and the audio stream that is captured by the microphone 114) and strip the audio components and the image components therefrom. The preprocessing system 170 is also configured to provide the audio and/or video components to various other components of the system 100, such as, for example, the machine learning devices 120, the audio processing system 150, the video processing system 160, and/or the like. In some embodiments, such as the embodiment depicted in FIG. 1, the preprocessing system 170 is a system that is separate and distinct from the other components of the system 100. In such embodiments, the preprocessing system 170 may include various hardware components such as, for example, processors, memory, data storage components (e.g., hard disc drives), communications hardware, display interface hardware, user interface hardware, and/or the like. In some embodiments, the preprocessing system 170 may be a service that is provided by a third-party provider. In other embodiments, the preprocessing system 170 may be a hardware component or software that is integrated with one or more other components of the system, such as, for example, the mobile device 110. For example, the mobile device 110 may include software thereon for preprocessing the video as it is captured in real time without having to transfer the video file to an external device for preprocessing.

It should be understood that while the user computing device 140 is depicted as a personal computer and the one or more machine learning devices 120, the data server 130, the audio processing system 150, the video processing system 160, and the preprocessing system 170 are depicted as servers, these are nonlimiting examples. More specifically, in some embodiments, any type of computing device (e.g., mobile computing device, personal computer, server, etc.) may be utilized for any of these components, or any one of these components may be embodied within other hardware components, or as a software module executed by other hardware components. Additionally, while each of these devices is illustrated in FIG. 1 as a single piece of hardware, this is also merely an example. More specifically, each of the components described with respect to the system 100 may represent a plurality of computers, servers, databases, etc.

FIG. 2A depicts illustrative internal components of the mobile device 110 of FIG. 1 that provide the mobile device 110 with the capabilities described herein. As depicted in FIG. 2A, the mobile device 110 may include a processing device 210, a non-transitory memory component 220, input/output (I/O) hardware 230, network interface hardware 240, a data storage component 250, user interface hardware 260, the imaging device 112, and/or the microphone 114. A local interface 200, such as a bus or the like, may interconnect the various components.

The processing device 210, such as a computer processing unit (CPU), may be the central processing unit of the mobile device 110, performing calculations and logic operations to execute a program. The processing device 210, alone or in conjunction with the other components, is an illustrative processing device, computing device, processor, or combination thereof. The processing device 210 may include any processing component configured to receive and execute instructions (such as from the data storage component 250 and/or the memory component 220).

The memory component 220 may be configured as a volatile and/or a nonvolatile computer-readable medium and, as such, may include random access memory (including SRAM, DRAM, and/or other types of random access memory), read only memory (ROM), flash memory, registers, compact discs (CD), digital versatile discs (DVD), and/or other types of storage components. The memory component 220 may include one or more programming instructions thereon that, when executed by the processing device 210, cause the processing device 210 to complete various processes, such as the processes described herein.

Referring to FIGS. 2A-2B, the programming instructions stored on the memory component 220 may be embodied as a plurality of software logic modules, where each logic module provides programming instructions for completing one or more tasks. Illustrative logic modules depicted in FIG. 2B include, but are not limited to, operating logic 280, sensor logic 282, data receiving logic 284, and/or data providing logic 286. Each of the logic modules depicted in FIG. 2B may be embodied as a computer program, firmware, or hardware, as an example.

The operating logic 280 may include an operating system and/or other software for managing components of the mobile device 110. The sensor logic 282 may include one or more programming instructions for directing operation of one or more sensors (e.g., the imaging device 112 and/or the microphone 114). Referring to FIGS. 1 and 2B, the data receiving logic 284 may generally include programming instructions for receiving data from one or more components external to the mobile device 110, such as, for example, data transmitted by the one or more machine learning devices 120, the data server 130, the user computing device 140, and/or the like. Still referring to FIGS. 1 and 2B, the data providing logic 286 may generally include programming instructions for transmitting data (e.g., videos, image portions thereof, and/or audio portions thereof) to one or more components external to the mobile device 110, such as, for example, data to the one or more machine learning devices 120, the data server 130, the user computing device 140, the audio processing system 150, the image processing system 160, the preprocessing system 170, and/or the like.

Referring again to FIG. 2A, the input/output hardware 230 may communicate information between the local interface 200 and one or more other components of the mobile device 110 not described herein. In some embodiments, the input/output hardware 230 may be used for one or more user interface components, including local user interface components and/or one or more remote user interface components. Alternatively or in addition, the user interface hardware 260 may include such components. For example, the user interface hardware 260 may include any component that can receive inputs from a user and translate the inputs to signals and/or data that cause operation of the mobile device 110 (e.g., a touchscreen interface, a keyboard, a mouse, and/or the like).

The network interface hardware 240 may include any wired or wireless networking hardware, such as a modem, LAN port, wireless fidelity (Wi-Fi) card, WiMax card, mobile communications hardware, and/or other hardware for communicating with other networks and/or devices. For example, the network interface hardware 240 may be used to facilitate communication between the various other components described herein with respect to FIG. 1.

The data storage component 250, which may generally be a storage medium, may contain one or more data repositories for storing data that is received and/or generated. The data storage component 250 may be any physical storage medium, including, but not limited to, a hard disk drive (HDD), memory, removable storage, and/or the like. While the data storage component 250 is depicted as a local device, it should be understood that the data storage component 250 may be a remote storage device, such as, for example, a server computing device, cloud based storage device, or the like. Illustrative data that may be contained within the data storage component 250 includes, but is not limited to, image data 252, audio data 254, and/or other data 256. The image data 252 may generally pertain to data that is captured by the imaging device 112, such as, for example, the image portion(s) of the video. The audio data 254 may generally pertain to data that is captured by the microphone 114, such as, for example, the audio portion(s) of the video. The other data 256 is generally any other data that may be obtained, stored, and/or accessed by the mobile device 110.

It should be understood that the components illustrated in FIGS. 2A-2B are merely illustrative and are not intended to limit the scope of this disclosure. More specifically, while the components in FIGS. 2A-2B are illustrated as residing within the mobile device 110, this is a nonlimiting example. In some embodiments, one or more of the components may reside external to the mobile device 110.

FIG. 3A depicts illustrative internal components of the one or more machine learning devices 120 of FIG. 1 that provide the one or more machine learning devices 120 with the capabilities described herein. As depicted in FIG. 3A, the one or more machine learning devices 120 may include at least one processing device 310, a non-transitory memory component 320, network interface hardware 340, input/output (I/O) hardware 330, and/or a data storage component 350. A local interface 300, such as a bus or the like, may interconnect the various components.

The processing device 310, such as a computer processing unit (CPU), may be the central processing unit of the one or more machine learning devices 120, performing calculations and logic operations to execute a program. The processing device 310, alone or in conjunction with the other components, is an illustrative processing device, computing device, processor, or combination thereof. The processing device 310 may include any processing component configured to receive and execute instructions (such as from the data storage component 350 and/or the memory component 320).

The memory component 320 may be configured as a volatile and/or a nonvolatile computer-readable medium and, as such, may include random access memory (including SRAM, DRAM, and/or other types of random access memory), read only memory (ROM), flash memory, registers, compact discs (CD), digital versatile discs (DVD), and/or other types of storage components. The memory component 320 may include one or more programming instructions thereon that, when executed by the processing device 310, cause the processing device 310 to complete various processes, such as the processes described herein.

Referring to FIGS. 3A-3B, the programming instructions stored on the memory component 320 may be embodied as a plurality of software logic modules, where each logic module provides programming instructions for completing one or more tasks. Illustrative logic modules depicted in FIG. 3B include, but are not limited to, operating logic 380, data receiving logic 384, data providing logic 386, machine learning logic 388, characteristic estimation logic 390, and/or stroke determination logic 394. Each of the logic modules depicted in FIG. 3B may be embodied as a computer program, firmware, or hardware, as an example.

The operating logic 380 may include an operating system and/or other software for managing components of the one or more machine learning devices 120. Referring to FIGS. 1 and 3B, the data receiving logic 384 may generally include programming instructions for receiving data from one or more components external to the one or more machine learning devices 120, such as, for example, data transmitted by the mobile device 110, the data server 130, the user computing device 140, the audio processing system 150, the image processing system 160, the preprocessing system 170, and/or the like. Still referring to FIGS. 1 and 3B, the data providing logic 386 may generally include programming instructions for transmitting data to one or more components external to the one or more machine learning devices 120, such as, for example, data to the mobile device 110, the data server 130, the user computing device 140, the audio processing system 150, the image processing system 160, the preprocessing system 170, and/or the like. Still referring to FIGS. 1 and 3B, the machine learning logic 388 may generally include programming instructions for executing one or more machine learning tasks. For example, the machine learning logic 388 may include programming instructions that correspond to one or more machine learning algorithms that are used by the one or more machine learning devices 120.

Still referring to FIGS. 1 and 3B, the characteristic estimation logic 390 may generally include one or more programming instructions for estimating one or more characteristics of a subject S from the image stream and/or the audio stream of the video captured by the mobile device 110. That is, the characteristic estimation logic 390 may include programming instructions for recognizing and indicating certain features of the subject S that are usable for the purposes of determining whether a stroke is occurring or has occurred, as described herein. Illustrative examples of features of the subject S that may be recognized using the characteristic estimation logic 390 may include certain facial features, certain movements, certain speech patterns, and/or the like, as described in greater detail herein. In addition, the stroke estimation logic 390 may generally include one or more programming instructions for estimating or determining whether a stroke has occurred, will occur, or is occurring based on the features recognized and indicated by the characteristic estimation logic 390.

Referring again to FIG. 3A, the input/output hardware 330 may communicate information between the local interface 300 and one or more other components of the one or more machine learning devices 120 not described herein. In some embodiments, the input/output hardware 330 may be used for one or more user interface components, including local user interface components and/or one or more remote user interface components.

The network interface hardware 340 may include any wired or wireless networking hardware, such as a modem, LAN port, wireless fidelity (Wi-Fi) card, WiMax card, mobile communications hardware, and/or other hardware for communicating with other networks and/or devices. For example, the network interface hardware 340 may be used to facilitate communication between the various other components described herein with respect to FIG. 1.

Still referring to FIG. 3A, the data storage component 350, which may generally be a storage medium, may contain one or more data repositories for storing data that is received and/or generated. The data storage component 350 may be any physical storage medium, including, but not limited to, a hard disk drive (HDD), memory, removable storage, and/or the like. While the data storage component 350 is depicted as a local device, it should be understood that the data storage component 350 may be a remote storage device, such as, for example, a server computing device, cloud based storage device, or the like. Illustrative data that may be contained within the data storage component 350 includes, but is not limited to, machine learning model data 352, characteristic data 354, and/or stroke estimation data 356. The machine learning model data 352 may generally pertain to data that is generated by the one or more machine learning devices 120 and/or data that is used for the purposes of generating a machine learning model. The characteristic data 354 is generally data that is obtained or generated with respect to the determined characteristics of the subject S (FIG. 1) based on the images and/or audio, as described herein. Still referring to FIG. 3A, the stroke estimation data 356 is generally data that is generated as a result of (or for the purposes of) estimating whether a stroke has occurred, will occur, or is occurring, as described herein.

It should be understood that the components illustrated in FIGS. 3A-3B are merely illustrative and are not intended to limit the scope of this disclosure. More specifically, while the components in FIGS. 3A-3B are illustrated as residing within the one or more machine learning devices 120, this is a nonlimiting example. In some embodiments, one or more of the components may reside external to the one or more machine learning devices 120.

Referring again to FIG. 1, the data stored by one or more components of the system 100 (e.g., the data server 130) may include a clinical dataset. The clinical dataset may be acquired from clinical subjects from any source. For example, a clinical dataset was acquired in the ERs of the Houston Methodist Hospital by the physicians and caregivers from the Eddy Scurlock Stroke Center at the Hospital under an IRB-approved study. The subjects chosen are subjects with suspicion of stroke or have already been identified with a high risk of stroke (transferred from other health providers) while visiting the ER. To help preserve the subjects' personal information, only limited information that is sufficient for generating the dataset was used to avoid any identifiable information to be collected and dispensed. The subjects are only assessed when they are in a relatively stable condition so the collection of data will not impose extra risk to very emergency cases. In some embodiments, the gender ratio of the clinical dataset is relatively balanced without intervention, and the race/ethnicity or age distributions are not manually controlled, which would roughly represent the real distribution of the incoming ER subjects.

Clinically, the ability of speech is an important and efficient indicator for the presence of stroke and is the preferable measurement doctors will use to make initial clinical impressions. For example, if a potential subject slurs, mumbles, or even fails to speak, he or she will have a very high chance of stroke. During the evaluation and recording stage, the NIH Stroke Scale is followed and the following speech tasks are performed on each subject: (1) to repeat a particular predetermined sentence, such as, for example, the following sentence “it is nice to see people from my hometown,” and (2) to describe a scene from a printed image, such as image 400 depicted in FIG. 4A showing a “cookie theft.” The “cookie theft” task requires the subject to retrieve, think, organize, and express the information, which will end up evaluating the subject's speech ability from both motor and cognitive aspects. Such an analysis has also been successful in identifying subjects with Alzheimer's-related dementia, aphasia, and some other cognitive-communication impairments, and would be suitable for stroke screening purposes.

To collect the data, the subjects are video recorded when they are performing the two tasks noted above. As depicted in FIG. 4B, the collection protocol is set up with the mobile device 110, such as, for example, an Apple® iPhone® such that the subject S is pointing the mobile device 110 towards his or her face F. Note that a mobile device stand or rack and/or an auxiliary microphone may be used if the subject S is too weak to hold the mobile device 110 stably. Each video is accompanied by metadata information on both clinical impressions by the caretaker (indicating the caretaker's initial judgment on whether the subject has a stroke or not from his/her speech and facial muscular conditions) and ground truth from a diffusion-weighted MRI (including the presence of acute ischemic stroke, transient ischemic attack (TIA), etc.).

One illustrative dataset includes 79 males and 72 females, non-specific of age, race/ethnicity, or the seriousness of stroke. Among the 151 individuals, 106 are subjects diagnosed with stroke using MRI, 45 are subjects who do not have a stroke but are diagnosed with other clinical conditions. A summary of demographics information is shown in Table 1 below. The diagnosing process described herein is formulated as a binary classification task and only attempts to identify stroke/TIA cases from non-stroke cases. Though there are varieties of stroke subtypes, binary output has been sufficient to work as the screening decision in certain clinical settings, such as the emergency room. However, it should be understood that the present disclosure is not solely related to a binary output. That is, in some aspects, the binary output may be supplemented with supplemental information such as explanatory information (e.g., a probability estimation, highlight of areas on the face that may show the signs of a stroke, highlight of segments of the speech that may show the signs of a stroke, etc.).

TABLE 1
Summary of subjects' demographic information in the dataset

| Attribute | Group | Stroke | Non-Stroke | Total |
|---|---|---|---|---|
| Gender | Male | 54 | 25 | 79 |
| | Female | 52 | 20 | 72 |
| Age | ≤65 y/o | 60 | 24 | 84 |
| | ≥65 y/o | 46 | 21 | 67 |
| Ethnicity | Hispanic | 7 | 4 | 11 |
| | Non-Hispanic | 95 | 39 | 134 |
| | Opt-out | 4 | 2 | 6 |
| Race | African-American | 34 | 9 | 43 |
| | Other | 72 | 36 | 108 |

| | | | |
|---|---|---|---|
| All subjects | 106 | 45 | 151 |

The dataset noted above is unique relative to other datasets because the cohort includes actual subjects visiting the ERs and the videos are collected under unconstrained, or “in-the-wild” conditions. Other experiments generally prepared experimental settings before collecting the image or video data, which results in uniform illumination conditions and minimum background noise. In the dataset noted above, the subjects can be in bed, sitting, or standing, where the background and illumination are usually not under ideal control conditions. Furthermore, other experiments enforce rigid constraints over the subjects' head motions, which avoids the alignment challenges and assumes stable face poses. In the present case, subjects were only asked to focus on the instructions, without rigidly restricting their motions, but using video processing methods to accommodate for the “in-the-wild” conditions. The acquisition of facial data in natural settings allows comprehensive evaluation of the robustness and practicability for real-world clinical use, remote diagnosis, and self-assessment in most settings.

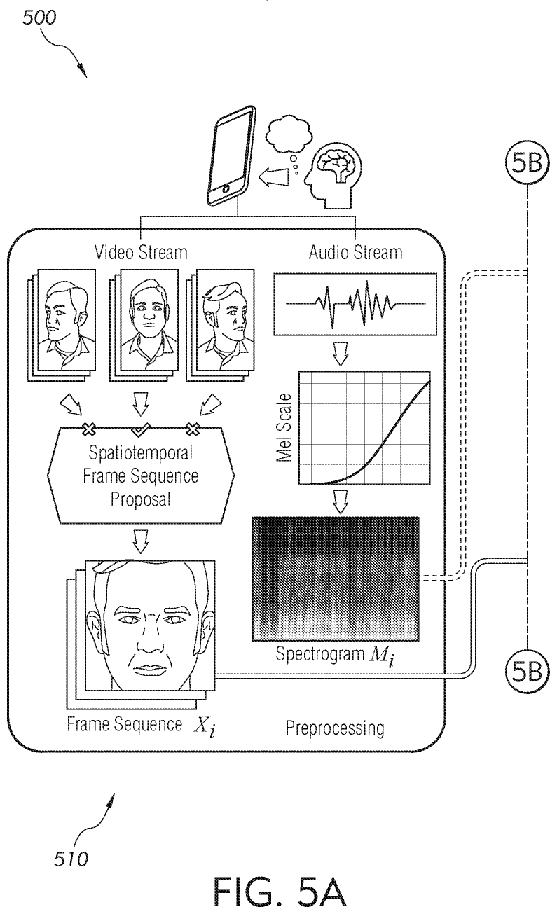

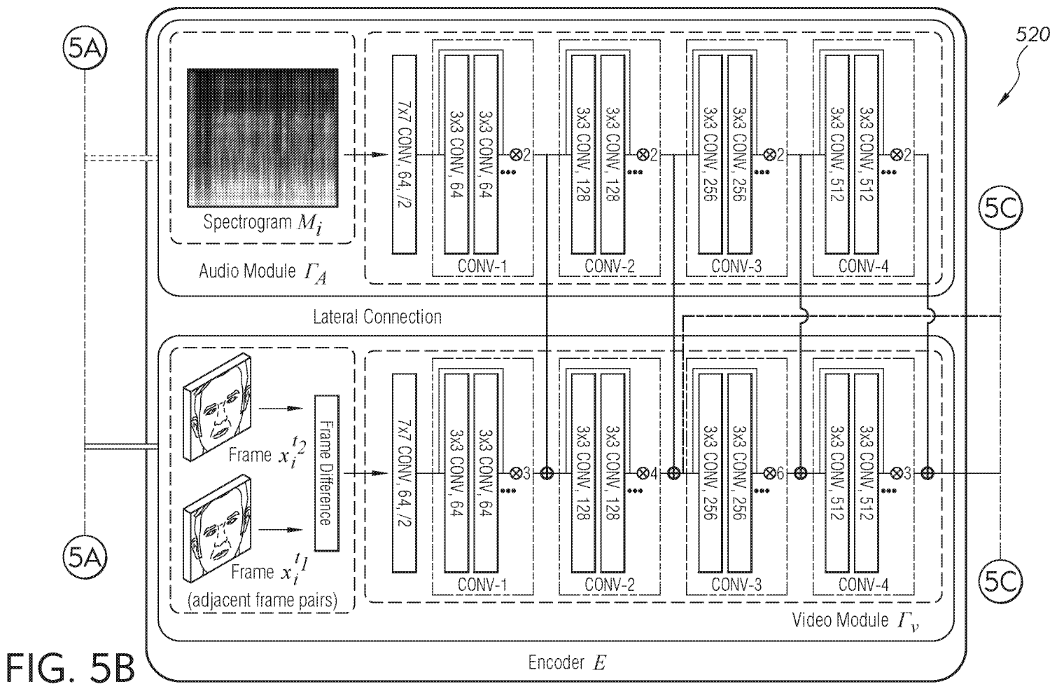

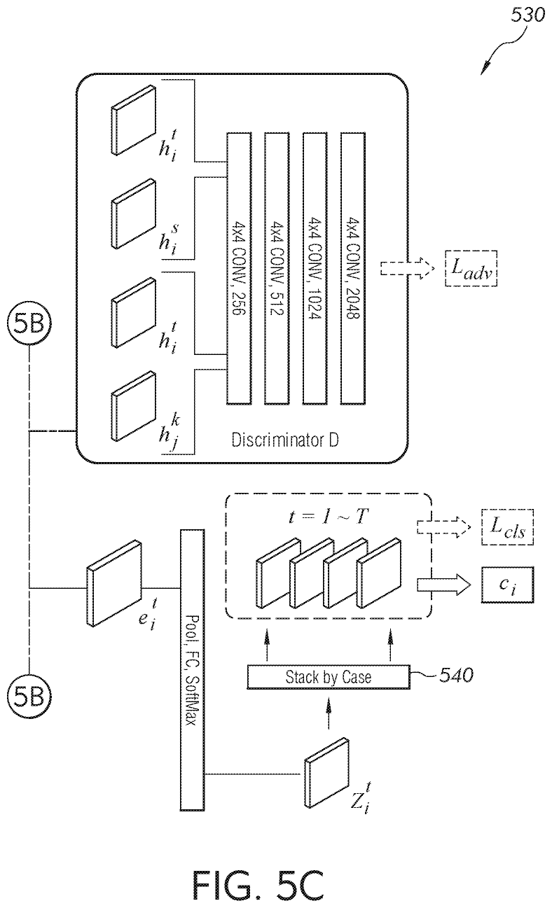

Referring now to FIGS. 5A-5C, a training framework 500 is depicted. The framework 500 includes a preprocessing portion 510, an encoder portion 520, and a discriminator portion 530. The preprocessing portion 510 involves a spatiotemporal facial frame sequence proposal and a transformation of audio feature to spectrograms. In the encoder portion 520, at any timestamp t, the input frame pairs xᵢ^{t}^{₁} and xᵢ^{t}^{₂} are adjacent pairs, and the total spectrogram is duplicated and appended as the audio features for time t. The symbol ⊕ indicates the concatenation with the lateral connection.

Initially, the i-th input raw video is preprocessed at the preprocessing portion 510 to obtain the facial-motion-only, near-frontal face sequence [symbol] ᵢ and its corresponding audio spectrogram [symbol] ᵢ. Then the audio-visual feature eᵢ is extracted from [symbol] ᵢ and [symbol] ᵢ by the lateral-connected dual-branch encoder portion 520, which includes a video module Γᵥ for local visual pattern recognition, and an audio module Γₐ for global audio feature analysis. The subject discriminator portion 530 is also employed to help the encoder portion 520 learn features that are insensitive to subject identity difference whereas sensitive to distinguish stroke from non-stroke. When training, the case-level label is used as a pseudo label for each video frame and train the framework as a frame-level binary classification model. The intermediate output feature maps from different videos to train the subject discriminator portion 530 and the encoder portion 520 adversarially. During inference, frame-level classification is performed, and then the case-level predictions are calculated at block 540 by averaging over all frames' probabilities to mitigate frame-level prediction noise. In some embodiments, the model may further incorporate other information (e.g., other available clinical information, patient demographic information) that is inputted.

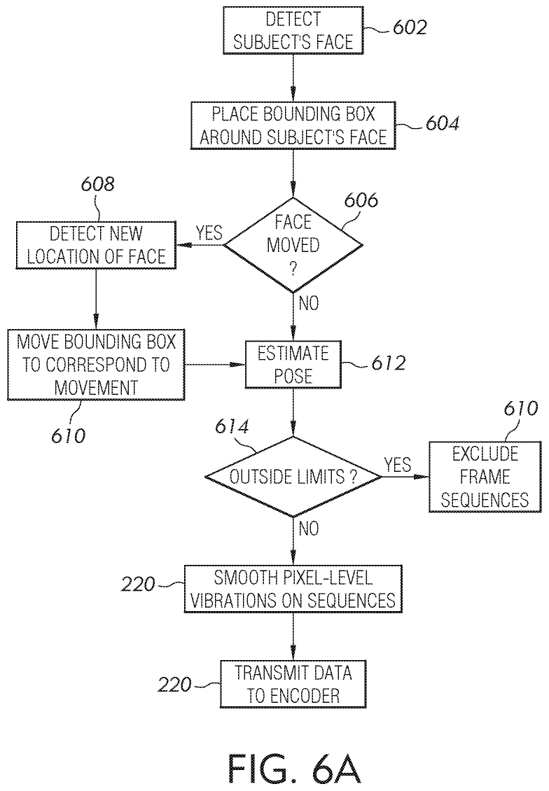

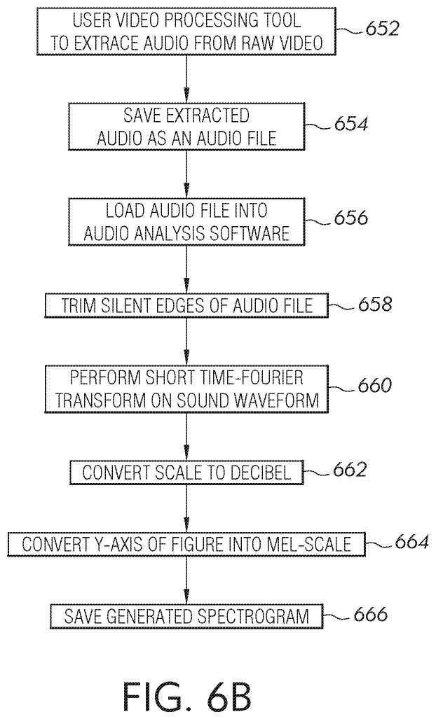

FIGS. 6A-6B depict a flow diagram of an illustrative data preprocessing process completed by the preprocessing portion 510 of FIG. 5A (which may be stored as process modules on one or more components of the system 100 depicted in FIG. 1, such as, for example, audio processing system 150, the image processing system 160, and/or the preprocessing system 170). For each raw video, a spatiotemporal proposal mechanism is used to extract frontal-face sequences from the raw video (FIG. 6A). For each audio extracted from the raw video, the soundtrack is transformed to a spectrogram that represents the amplitude at each frequency level over time (FIG. 6B).

FIG. 6A depicts a flow diagram relating to spatiotemporal proposal of facial action video. In facial motion analysis, one challenge is to achieve good face alignment. As the data described herein tend to be more “in-the-wild,” a pipeline is introduced to extract frame sequences with near-frontal facial pose and minimum non-facial motions. At block 602, a subject's face is detected with a face detection algorithm. A rigid, square bounding box is placed around the subject's face at block 604. At block 606, a determination is made as to whether the subject's face has moved. If so, the process moves to blocks 608 and 610 before proceeding to block 612. If not, the process moves directly to block 612. At block 608, a new location of the face is detected and at block 610, the bounding block is moved to correspond to the new location of the face. The processes described with respect to blocks 602-610 are generally completed using a face detection and tracking algorithm, such as for example, dlib (provided by Davis King). At block 612, the pose of the subject's face is estimated. At block 614, a determination is made as to whether the frame sequences are outside various limits. For example, frame sequences 1) estimated with significant roll, yaw, or pitch, 2) showing continuously changing pose metrics, or 3) having excessive head translation or too little change estimated with optical flow magnitude with the previous frame are considered to be outside limits. If the frame sequences are outside the limits (block 614: YES), the process moves to block 616 where they are excluded (detailed criteria is presented below). If the frame sequences are not outside the limits (block 614: NO), the process moves to block 618. At block 618, a video stabilizer with a sliding window over the trajectory of between-frame affine transformations smooths out pixel-level vibrations on the sequences The face tracking process and frame selection process will expect some variation of regions captured by the algorithm, which will result in a tiny change/vibration of the subject's face location. To mitigate such vibrations, the video stabilization is performed over the frame sequences. The between-frame changes in the sequence are regarded as a continuous function that takes different values over time. Larger between-frame changes correspond to higher function value. Here, affine transform between the two consecutive frames is considered and the affine parameters are estimated as the function value. To reduce the abrupt changes within nearby frames while maintaining the overall trends of motion, the average of the between-frame affine parameters over the coming batch (i.e., 30) of frames is calculated and the value is used as the current transform. That is, the “sliding window” average transformations that makes the transformation function values smoother over time. At block 620, the data corresponding to the frame sequences are passed to the encoder 520 (FIG. 5B).

FIG. 6B depicts a flow diagram relating to speech spectrum analysis. Instead of transcribing the vocal audio to text corpus, which may suffer from translation errors, spectrograms for speech analysis are used for the following reasons: (1) a spectrogram is the complete representation of an audio file since the amplitude over frequency bands are captured over time; and (2) by choosing a spectrogram, which is image-like input, similar networks can be adapted for the two branches to ensure they have rather similar training dynamics to converge at a similar pace, which will make them cooperate better. A spectrogram can be generated using a commercially available software program, such as, for example, librosa (McFee, Brian, Colin Raffel, Dawen Liang, Daniel P W Ellis, Matt McVicar, Eric Battenberg, and Oriol Nieto. “librosa: Audio and music signal analysis in python.” In Proceedings of the 14th python in science conference, pp. 18-25. 2015). The Mel Scale and Fast Fourier Transform (FFT) are used to transform the audio signal before plotting the spectrogram. As depicted in FIG. 6B, the process to create a Mel spectrogram includes using a video processing tool (e.g., FFMPEG) to extract the audio from raw video at block 652. The extracted audio is saved as an audio file (e.g., a .wav file) having a particular bit rate and frequency (e.g., a 160k bit rate and 44100 Hz frequency) at block 654. An audio analysis software package (e.g., librosa) is used to load the extracted audio file at block 656 and trim the silent edges at block 658. At block 660, short-time Fourier transform is performed on the sound waveform, and at block 662, the scale is converted to decibel. The y-axis of the figure is then converted into the Mel-scale at block 664 and the generated spectrogram is visualized and saved at block 666.

As depicted in FIGS. 5B-5C, after preprocessing the raw input videos, stroke-discriminative and identity-independent features are extracted from the input video and audio via the feature encoder 520 and the subject discriminator 530.

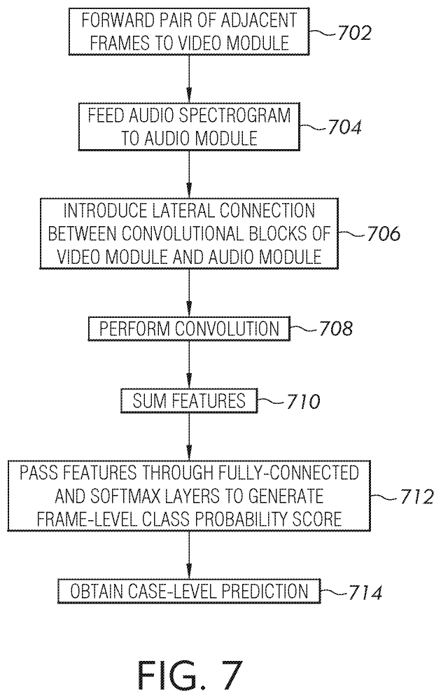

FIG. 7 depicts an illustrative process of extracting features. Let [symbol] ᵢ=(xᵢ¹, . . . , xᵢ^{T}), which denotes a sequence of T temporally-ordered frames from the i-th input video and its corresponding spectrogram is [symbol] ᵢ. The features are extracted through feature encoder 520 (FIG. 5B), which includes one video module Γᵥ and one audio module Γₐ, fused by lateral connection.

At block 702, to extract temporal visual feature from the input [symbol] ᵢ, a pair of adjacent frames from [symbol] ᵢ is forwarded to the video module Γᵥ. Due to the frame-proposal process, the original frame sequence sometimes has long gaps between two nearby frames, which will result in large, non-facial differences being captured. This is addressed by keeping track of frame index and only sample a specific number of real adjacent frame pairs in [symbol] ᵢ (frame xᵢ^{t}^{₁} and xᵢ^{t}^{₂}) to extract local visual information. Instead of directly inputting a pair of frames, for better capturing subtle facial motions between adjacent frames, the image difference between xᵢ^{t}^{₁} and xᵢ^{t}^{₂} is computed and passed through the network as the feature for the frame pair xᵢ^{t}.

At block 704, to extract disease patterns from the input audio spectrogram [symbol] ᵢ, the audio spectrogram [symbol] ᵢ is fed to the audio module Γₐ. Since [symbol] ᵢ contains the whole temporal dynamics of input audio sequence, [symbol] ᵢ is appended to each frame pair xᵢ^{t} and xᵢ^{t+1} to provide global context for the frame-level stroke classification.

At block 706, to effectively combine features of video [symbol] ᵢ and audio [symbol] ᵢ at different levels, lateral connection is introduced between the convolutional blocks of the video module Γᵥ and the audio module Γₐ. To ensure the features are aligned when being appended, 1×1 convolution is performed at block 708 to project the global audio feature to the same embedding space as the local frame features and at block 710, the features are summed. Compared with the late fusion of two branches that may be used, lateral connections-based fusion not only combines more levels of features, but also enables different branch dynamics to stay similar, which will maintain the convergence rate of each branch to be relatively closer and help the global context better complement the local context during the training stage. The final fused features eᵢ^{t} are passed through the fully-connected and softmax layers to generate the frame-level class probability score zᵢ^{t} at block 712. The case-level prediction cᵢ is obtained at block 714 by stacking and averaging the frame-level predictions.

Referring again to FIGS. 5B and 5C, due to the relatively small number of available videos, it is easy and tempting for the encoder 520 to memorize the facial and audio features of each subject and just match testing subjects with training subjects based on similarity in appearance and voice when performing the inference. To avoid this issue of classification based on subject-dependent features, the subject discriminator 530 is designed to help the encoder 520 learn subject-independent features. The discriminator 530 is designed to simply distinguish whether the input pair of intermediate features from the encoder 520 are from the same subject or not and will be used to adversarially train the encoder 520.

In theory, the framework described herein can employ various networks as its backbone. For example, ResNet-34 for Γ_{y} and ResNet-18 for Γₐ may be used to accommodate for the relatively small size of video dataset, the simplicity of the spectrogram, and to reduce the computational cost of the framework. For the discriminator 530, four (4) convolution layers are used with a binary output. For the intermediate feature input to the discriminator, the output feature is taken from the second block (CONV-2) of the ResNet-34 after the lateral connection at this level. The choice of intermediate feature is ablated, as described in greater detail herein.

For training of the framework described herein, the following loss function is employed:

Classification loss: To help E learn stroke-discriminative features, a standard binary cross-entropy loss between the prediction zᵢ^{t} and video label [symbol] ᵢ for all the training videos and their T frames:

$${ℒ}_{cls} ( E ) = - {\sum }_{i} {\sum }_{t} ( {i}_{} log {z}_{i}^{t} + ( 1 - {i}_{} ) ( 1 - log {z}_{i}^{t} ) ) \qquad ( 1 )$$

Adversarial loss: To encourage the encoder E to learn subject-independent features, a novel adversarial loss is introduced to ensure that the output feature map hᵢ^{t} does not carry any subject-related information. The adversarial loss is imposed via an adversarial framework between the subject discriminator 530 and the feature encoder 520, as shown in FIG. 5B. The encoder 520 provides a pair of feature maps, either computed from the same subject video (hᵢ^{t}, hᵢ^{s}) at time t and s, or from different subject videos (hᵢ^{t}, hⱼ^{k}) at time t and k, where time s and k can be randomly chosen. The discriminator 530 attempts to classify the pair as being from the same/different subject video using a l₂ loss, as LS-GAN adopts:

$${ℒ}_{adv} ( D ) = - {\sum }_{i} {\sum }_{t} ( {\vert D ( {h}_{i}^{t} , {h}_{i}^{s} ) \vert}_{2} + {\vert 1 - D ( {h}_{i}^{t} , {h}_{j}^{k} ) \vert}_{2} ) \qquad ( 2 )$$

The adversarial framework further imposes a loss function on the feature encoder 520 that tries to maximize the uncertainty of the discriminator 530 output on the pair of frames:

$${ℒ}_{adv} ( E ) = - {\sum }_{i} {\sum }_{t} ( {\vert \frac{1}{2} - D ( {h}_{i}^{t} , {h}_{i}^{s} ) \vert}_{2} + {\vert \frac{1}{2} - D ( {h}_{i}^{t} , {h}_{j}^{k} ) \vert}_{2} ) \qquad ( 3 )$$

Thus the encoder 520 is encouraged to produce features that the discriminator 530 is unable to classify if they come from the same subject or not. In so doing, the features h cannot carry information about subject identity, thus avoiding the model to perform inference based on subject-dependent appearance/voice features. Note that the model is different from classic adversarial training used since the focus is on classification and there is no generator network in the framework described herein. In some embodiments, the model may further be trained based on other information (e.g., other available clinical information, patient demographic information) that is inputted.

Overall training objective: During training, the sum of the above losses is minimized:

l= [symbol] _{cls}(E)+λ( [symbol] _{adv}(E)+ [symbol] _{adv}(D)) (4)

where λ is the balancing parameter. The first two terms can be jointly optimized, but the discriminator 530 is updated while the encoder 520 is held constant.

When testing, because the model described herein is trained with pseudo frame-level labels, to mitigate frame-level prediction noise, perform frame-level classification is first performed using the encoder 520, and the case-level prediction is calculated by summing and normalizing class probabilities over all frames, and the predicted case label will be the class with higher probability.

To better present the details of the methods described herein, the setup and implementation details are first introduced and then the comparative study with baselines are shown. In addition, the power of model components and structures are ablated to validate the design.

For setup and implementation, The whole framework is running on Python 3.7 with Pytorch 1.1, OpenCV 3.4, CUDA 9.0, and Dlib 19. Both ResNet models are pre-trained on ImageNet. Each .mov file from the mobile device 110 (FIG. 1) is first separated as frame sequences as batches of .png files and one global .wav audio file.

For the frame sequences, the location of the subject's face is detected as a square bounding box with Dlib's face detector and track it using the Python implementation of ECO tracker. Pose estimation by solving a direct linear transformation from the 2D landmarks predicted by Dlib to a 3D ground truth facial landmarks of an average face, which resulted in three values corresponding to the angular magnitudes over three axes (pitch, roll, yaw). To tolerate estimation errors, faces with angular motions less than a threshold β₁ are marked as frontal faces. A 5-frame sliding window records the between-frame changes (three differences) in the pose. If the total changes (3 axes) sum up to more than a threshold β₂, the frames are abandoned starting from the first position of the sliding window. In the meantime, the between-frame changes are measured by optical flow magnitude. If the total estimated change is smaller than β₁ (no motion) or larger than βₕ (non-facial motions), the frame is excluded. β₁=5°, β₂=20°, βₗ=0.01 and βₕ=150 is empirically set. After manipulation, the size of each frame in the videos is cropped to 224×224×3 to align with the ImageNet dataset. The real frame numbers are also kept to ensure that only adjacent frame pairs are loaded to the network.

For the audio files, librosa may be used for loading and trimming, and plot the Log-Mel spectrogram, where the horizontal axis is the temporal line, the vertical axis is the frequency bands, and each pixel shows the amplitude of the soundwave at the specific frequency and time. The output spectrogram is also set to the size of 224×224×3.

The entire pipeline is trained on a personal computer with a quad-core CPU, 16 GB RAM, and a NVIDIA GPU with 11 GB VRAM. To accommodate for the class imbalance inside the dataset and ER setting, a higher class weight (2.0) is assigned to the non-stroke class. In evaluation, the accuracy, specificity, sensitivity, and area under the ROC curve (AUC) from 5-fold cross-validation results is referred to over the full dataset. The learning rate is tuned to 1e-5 and an early stop is completed at epoch 8 due to the quick convergence of the network. The batch size is set to be 64 and λ in (4) is set to be 10. The same parameters are also applied to the baselines and the ablation studies.

Table 2 below depicts comparative study results with a baseline. Raw is results with a threshold of 0.5 after the final softmax output and aligned is results with the threshold that makes the specificity aligned. The best values are in bold text:

TABLE 2

| | Metrics | | | | | | |
|---|---|---|---|---|---|---|---|
| | Accuracy (%) | | Specificity (%) | | Sensitivity (%) | | AUC |
| Method | Raw | Aligned | Raw | Aligned | Raw | Aligned | — |
| Module Γₐ (Audio) | 60.93 | 46.36 | 22.22 | 53.33 | 77.36 | 43.40 | 0.5168 |
| Module Γᵥ (Video) | 70.86 | 52.31 | 13.13 | 53.33 | 95.28 | 51.89 | 0.5381 |
| I3D (Video) | 62.25 | 42.38 | 15.55 | 53.33 | 82.08 | 37.73 | 0.4419 |
| SlowFast (Video) | 67.55 | 36.42 | 2.22 | 53.33 | 95.28 | 29.25 | 0.3981 |
| MMDL (Audio + Video) | 70.20 | 58.27 | 20.00 | 53.33 | 91.50 | 60.38 | 0.5893 |
| DSN (Audio + Video) | 68.21 | 63.56 | 37.77 | 53.33 | 81.13 | 67.92 | 0.6631 |
| Clinical Impression (CI) | 58.94 | | 53.33 | | 61.32 | | 0.5733 |

Baseline models for both video and audio tasks are constructed. For each video/audio, the ground truth for comparison is the binary diagnosis result obtained through the MRI scan. The chosen baselines are introduced as follows:

Audio Module Γₐ. The first corresponding baseline is the strip audio module from the proposed method that takes the preprocessed spectrograms as input. The same setup is used to train the audio module and obtain binary classification results on the same data splits.

Video Module Γᵥ. The other baseline is the strip video module from the proposed method that takes the preprocessed frame sequences as input. The same adversarial training scheme is used to train the video module and obtain binary classification results on the same data splits.

I3D. The Two-Stream Inflated 3D ConvNet (I3D) expands filters and pooling kernels of 2D image classification ConvNets into 3D to learn spatiotemporal features from videos. I3D can be inferior due to the calculation of optical flow can be time-consuming and results in more noises.

SlowFast. The SlowFastnetwork is a video recognition network proposed by Facebook Inc. that involves (i) a slow pathway to capture spatial semantics, and (ii) a fast pathway to capture motion at fine temporal resolution. SlowFast achieves strong performance on action recognition in video and has been a powerful state-of-the-art model in recent years.

MMDL. MMDL is a preliminary version of the proposed two-branch method that takes similar preprocessed frame sequences for the video branch, but text transcripts for the audio branch. The video branch uses feature difference instead of image difference (which will be ablated later), and the audio branch was an LSTM that performs text classification. Due to drastically different network structures, the two branches only have connections in the final layer using a “late-fusion” scheme.

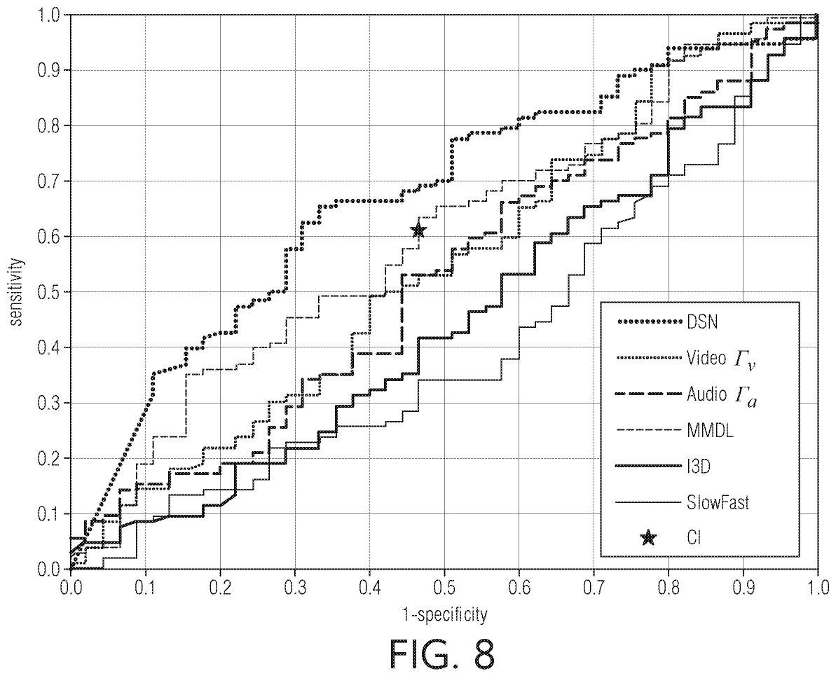

Because the objective herein is to perform the stroke screening for incoming subjects, the methods described herein and the baselines are compared to the ER doctors' clinical impression we obtained with the metadata information. The effectiveness of the methods described herein are examined with clinical impression and baselines by aligning the specificity of each method to be the same through changing the threshold for binary cutoffs, while checking and comparing for other measurements. The results are shown in Table 3 below. For better comparison, the ROC curves for the model described herein and baselines are also plotted in FIG. 8, together with the clinical impression from the physicians (represented by the star in FIG. 8). The x-coordinate represents the sensitivity or false positive rate (FPR), and the y-coordinate is 1-specificity or true positive rate (TPR). That is, the ROC curve shows the performance of a binary classifier when the decision boundary is varied. When the ROC curve for some classifiers can more towards the upper left corner than others, the performance of the classifier is considered better. In the ROC curve plot depicted in FIG. 8, the framework described herein has the highest performance as the curve is the most toward upper left. It also beats the performance of clinicians that is indicated by the green point.

From both Table 2 above and FIG. 8, it is evident that the framework described herein outperforms several state-of-the-art methods as well as its single module variants. The framework described herein improves specificity by 17.77% and AUC by 0.0738 over previous frameworks. This is a major improvement considering the prior work has already demonstrated good performance on the preliminary version of the dataset. The improvements came from the following aspects: i) firstly, by adopting a different audio representation and introducing the lateral connections, the framework described herein resolves the unstable convergence problem in the prior work caused by different training dynamics of branches, while also sharing low- and high-level features for a better combination of global audio context and local frame features; ii) secondly, the adversarial training scheme can keep the network from remembering the identity features and extract pure stroke-related features for the network to learn, which also mitigates the overfitting problem. The two aspects are discussed in the following ablation studies; iii) in another aspect, using spectrograms instead of the text transcripts will maximally preserve the patterns in the original audio files since the soundwave information is fully presented without inference, addition, or deletion.

Moreover, comparing the Video Module Γᵥ with two state-of-the-art video recognition models, a much higher model performance is observed. When specificity is aligned, Video Module Γᵥ is achieving 9.93% higher accuracy, 14.16% higher sensitivity, and 0.0972 higher AUC than I3D and 15.89% higher accuracy, 22.64% higher sensitivity and 0.14 higher AUC than SlowFast. When compared with ER physicians, the framework described herein shows 0.0898 higher AUC value, and 6.6% higher sensitivity, and 4.62% higher accuracy when aligned the same specificity with clinicians, which illustrating its practicability and effectiveness.

Through the experiments, all the methods are experiencing low specificity (i.e., identifying non-stroke cases), which is reasonable because the subjects have a suspicion of stroke or show neurological disease-related patterns, rather than the general public. The nature of the task completed herein is much harder than previous works.

As new subjects are continuously added to the cohort, the dataset may become more and more diverse and even more challenging for the clinicians (for the original dataset, the performance of the clinicians was 72.94% accuracy, 77.78% specificity, 70.68% sensitivity, and 0.7423 AUC). This may be due to the addition of a decent number of hard cases, where the patterns for stroke are too subtle for the clinicians to capture. Even so, when specificity has been aligned, the method described herein still outperforms the clinicians. ER doctors tend to rely more on the speech abilities of the subjects and may have difficulty in cases with too subtle facial motion incoordination. We infer that the video module in our framework can detect those subtle facial motions that doctors can neglect and complement the diagnosis based on speech/audio. The drop in the specificity is regarded as permissible comparing to the improvements in sensitivity, since in stroke screening, failing to spot a subject with stroke (false-negative) will end up with very serious results. On the other hand, when making the decisions, the ER doctors have access to emergency imaging reports and other information in the Electronic Health Records (EHR) besides the video and audio information.

The running time of the proposed approach is analyzed. The recording runs for a minute, the extraction of audio and generation of spectrograms takes an extra minute, and the video processing is completed in three minutes. The prediction with the deep models can be achieved within half a minute on a desktop with NVIDIA GTX 1070 GPU. Therefore, the evaluation process takes no more than six minutes per case. More importantly, the process is almost running at zero external cost and would be contactless, not harming the subjects with equipment or by radiation. Considering a complete MRI scan will take more than an hour to perform, a specialized device to run, and hundreds of dollars charged, the proposed method is more ideal for performing both cost and time-efficient stroke assessments in an emergency setting.

The approach described herein may be clinically relevant and can be deployed effectively on smartphones for fast and accurate assessment of stroke by caregivers such as ER doctors, at-risk subjects, or other caregivers. If the approach is further optimized and deployed onto a smartphone, the spatiotemporal face frame can be performed and the speech audio processing on the mobile device 110 (FIG. 1). Cloud computing can be leveraged to perform the prediction in no more than a minute after the frames are compressed and uploaded. In such a case, the total time for one assessment should be within three to four minutes. Moreover, with minimal user education required, such a framework can allow for the subjects' self-assessments even before the ambulance arrives. With different labeling of data, the pipeline is also valuable in the screening of other oral-facial neurological conditions.

### Example

### Emergency Room Subject Dataset

The clinical dataset for this study was acquired in the emergency rooms (ERs) of Houston Methodist Hospital by the physicians and caregivers from the Eddy Scurlock Stroke Center at the Hospital under an IRB-approved study. We took months to recruit a sufficiently large pool of subjects in certain emergency situations. The subjects chosen are subjects with suspicion of stroke while visiting the ER. 47 males and 37 females have been recruited in a race-nonspecific way.

Each subject is asked to perform two speech tasks: 1) to repeat the sentence “it is nice to see people from my hometown” and 2) to describe a “cookie theft” picture. The ability of speech is an important indicator to the presence of stroke; if the subject slurs, mumbles, or even fails to repeat the sentence, they have a very high chance of stroke. The “cookie theft” task has been used in neuropsychiatric training in identifying subjects with Alzheimer's-related dementia, aphasia, and other cognitive-communication impairments.

The subjects are video recorded as they perform the two tasks with an iPhone X's camera. Each video has metadata information on both clinical impressions by the ER physician (indicating the doctor's initial judgement on whether the subject has a stroke or not from his/her speech and facial muscular conditions) and ground truth from the diffusion-weighted MRI (including the presence of acute ischemic stroke, transient ischemic attack (TIA), etc.). Among the 84 individuals, 57 are subjects diagnosed with stroke using the MRI, 27 are subjects who do not have a stroke but are diagnosed with other clinical conditions. In this work, we construct a binary classification task and only attempt to identify stroke cases from non-stroke cases, regardless of the stroke subtypes.

Our dataset is unique, as compared to existing ones, because our subjects are actual subjects visiting the hospitals and the videos are collected in an unconstrained, or “in-the-wild”, fashion. In most existing work, the images or videos were taken under experimental settings, where good alignment and stable face pose can be assumed. In our dataset, the subjects can be in bed, sitting, or standing, where the background and illumination are usually not under ideal control conditions. Apart from this, we only asked subjects to focus on the picture we showed to them, without rigidly restricting their motions. The acquisition of facial data in natural settings makes our work robust and practical for real-world clinical use, and ultimately empowers our method for remote diagnosis of stroke and self-assessment in any setting.

### Methodology

We propose a computer-aided diagnosis method to assess the presence of stroke in a subject visiting ER. This section introduces our information extraction methods, separate classification modules for video and audio, and the overall network fusion mechanism.

### Information Extraction

For each raw video, we propose a spatiotemporal proposal of frames and conduct a machine speech transcription for the raw audio.

Spatiotemporal proposal of facial action video: We develop a pipeline to extract frame sequences with near-frontal facial pose and minimum non-facial motions. First, we detect and track the location of the subject's face as a square bounding box. During the same process, we detect and track the facial landmarks of the subject, and estimate the pose. Frame sequences 1) with large roll, yaw or pitch, 2) showing continuously changing pose metrics, or 3) having excessive head translation estimated with optical flow magnitude are excluded. A stabilizer with sliding window over the trajectory of between-frame affine transformations smooths out pixel-level vibrations on the sequences before classification.

Speech transcription: We record the subject's speech and transcribe the recorded speech audio file using Google Cloud Speech-to-Text service. Each audio segment is turned into a paragraph of text in linear time, together with a confidence score for each word, ready for subsequent classification.

### Deep Neural Network for Video Classification

Facial motion abnormality detection is essential to stroke diagnosis, but challenges remain in several approaches to this problem. First, the limited number of videos prevent us from training 3D networks (treating time as the 3rd dimension) such as C3D and R3D, because these networks have a large number of parameters and their training can be difficult to converge with a small dataset. Second, although optical flow has been proven useful in capturing temporal changes in gesture or action videos, it is ineffective in identifying subtle facial motions due to noise and can be expensive to compute. Network architecture: In this work, we propose the deep neural network shown in FIG. 2 for binary classification of a video to stroke vs. non-stroke. For a video consisting of N frames, we take the k^{th} and k+1^{th} frames and calculate the difference between their embedding features right after the first 3×3 convolutional layer. Next, a ResNet-34 model outputs the class probability vector pₖ for each frame based on the calculated feature differences. An overall temporal loss L is calculated by combining a negative log-likelihood (NLL) loss term and smoothness regularization loss terms based on the class probability vectors of three consecutive frames. From the frame-level class probabilities, a video-level class probability vector is obtained by averaging over all frames' probabilities, and the predicted video label will be the class with higher probability.

Relation embedding: One novelty of our proposed video module is in the classification using feature differences between consecutive frames instead of using directly the frame features. The rationale behind this choice is in that we expect the network to learn and classify based on motion features. Features from single frames contain a lot of information about the appearance of face which are useful for face identification but not useful in characterizing facial motion abnormality.

Temporal loss: Denote i as the frame index, yᵢ as the class label for frame i obtained based on the ground truth video label, and pᵢ as the predicted class probability. The combined loss L for frame i is defined with three terms: L⁽ⁱ⁾=L₁⁽ⁱ⁾+α(L₂_{₍₁₎}⁽ⁱ⁾+βL₂_{₍₂₎}⁽ⁱ⁾) where α and β are tunable weighting coefficients. The first loss term, L₁⁽ⁱ⁾=−(yᵢ log pᵢ+(1−yᵢ) log(1−pᵢ)), is the NLL loss. Note that we sample a subset of the frames to mitigate the overfitting on shape identity. By assuming consecutive frames have continuous and similar class probabilities, we develop a L₂_{₍₁₎}⁽ⁱ⁾ loss defined on the frame class probabilities for three adjacent frames where A is a small threshold to restrict the inconsistency by penalizing those frames with large class probability differences. We also design another loss, L₂_{₍₂₎}⁽ⁱ⁾, to encourage random walk around the convergence point to mitigate overfitting. Specifically,

$${L}_{{2}_{( 1 )}}^{( i )} = {\sum }_{j ∈ [ 0 , 1 , 2 ]} max \{ ( {\vert p}_{i + j} - {p}_{i + j + 1} \vert - λ ) , 0 \} \qquad ( 1 )$$

$${L}_{{2}_{( 2 )}}^{( i )} = {\sum }_{j ∈ [ 0 , 1 , 2 ]} {II}_{{p}_{i + j} = {p}_{i + j + 1}} \qquad ( 2 )$$

In practice, we adopt a batch training method, and all frames in a batch are weighted equally for loss calculation.

### Transcript Classification for Speech Ability Assessment

We formulate the speech ability assessment as a text classification problem. Subjects without speech disorder complete the speech task with organized sentences and maintain a good vocabulary set size, whereas subjects with speech impairments either put up a few words illogically or provide mostly unrecognizable speech. Hence, we concurrently formulate a binary classification on the speech given by the subjects to determine if stroke exists.

Preprocessing: For each speech case T:={tᵢ, . . . , t_{N}} extracted from the obtained transcripts where tᵢ is a single word and N is the number of words in the case, we first define the encoding of the words E over the training set by their order of appearance, E(tᵢ):=dᵢ, dᵢ∈I; E(T):=D and D={dᵢ, . . . , d_{N}}∈Iⁿ. We denote the vocabulary size obtained as v. Due to the length difference between cases, we pad the sequences to the max length m of the dataset, so that D′={dᵢ, . . . , d_{N}, p₁, . . . , pₘ₋ₙ}∈I^{m} where pᵢ denotes a constant padding value. We further embed the padded feature vectors to an embedding dimension E so that the final feature vector has X:={xᵢ, . . . , xₘ} and xᵢ∈R^{E×v}.

Text classification with Long Short-Term Memory (LSTM): We construct a basic bidrectional LSTM model to classify the texts. For the input X={xᵢ, . . . , xₘ}, the LSTM model generates a series of hidden states H:={h₁, . . . , hₘ} where hᵢ∈R^{t}. We take the output from the last hidden state hₜ, apply a fully-connected (FC) layer before output (class probabilities/logits)ŷᵢ∈R². For our task, we leave out the last FC layer for model fusion.

### Two-Stream Network Fusion.

Overall structure of the model: FIG. 3 shows the data pipeline of the proposed fusion model. Videos that are collected following the protocol are uploaded to a database, while audios are extracted and forwarded to Google Cloud Speech-to-Text which generates the transcript. Meanwhile, videos are sent to the spatiotemporal proposal module to perform face detection, tracking, cropping, and stabilization. The preprocessed data are further handled per-case-wise and loaded into the audio and video modules. Finally, a “meta-layer” combines the outputs of the two modules and gives the final prediction on each case.

Fusion scheme: We take a simple fusion scheme of the two models. For both the video and text/audio modules, we remove the last fully-connected layer before the output and concatenate the feature vectors. We construct a fully-connected “meta-layer” for the output of class probabilities. For all the N frames in a video, the frame-level class probabilities from the model are concatenated into Ŷ={ [symbol] , . . . , [symbol] }. The fusion loss LF is defined in a similar way as the temporal loss; instead of using only video-predicted probabilities, the fusion loss combines both video- and text-predicted probabilities. Note again that the fusion model operates at the frame level, and a case-level prediction is obtained by summing and normalizing class probabilities over all frames.

### Experiments and Results

Implementation and training: The whole framework is running on Python 3.7 with Pytorch 1.4, OpenCV 3.4, CUDA 9.0, and Dlib 19. The model starts with a pretrained model on ImageNet. The entire pipeline runs on a computer with a quad-core CPU and one GTX 1070 GPU. To accommodate for the existing class imbalance inside the dataset and ER setting, a higher class weight (1.25) is assigned to the non-stroke class. For evaluation, we report the accuracy, specificity, sensitivity and area under the ROC curve (AUC) from 5-fold cross validation results. The loss curves for one of the folds are presented in FIG. 4. The learning rate is tuned to 0.0001 and we early stop at epoch 8 due to the quick convergence and overfitting issue. It is worth mentioning that the early stop strategy and the balanced weight are applied to the baselines below.

Baselines and comparison: To evaluate our proposed method, we construct a number of baseline models for both video and audio tasks. The ground truth for comparison is the binary diagnosis result for each video/audio. General video classification models for video tagging or action recognition are not suitable for our task since they require all frames throughout a video clip to have the same label. In our task, since stroke subjects may have many normal motions, the frame-wise labels may not be equal to the video label all the time. For single frame models such as ResNet-18, we use the same preprocessed frames as input and derive a binary label for each frame. The label with more frames is then assigned as the video-level label. For multiple frame models, we simply input the same preprocessed video. We also compare with a traditional method based on identifying facial landmarks and analyzing facial asymmetry (“Landmark+Asymmetry”), which detects the mid-line of a subject's face and checks for bilateral pixel-wise differences on between-frame optical-flow vectors. The binary decision is given by statistical values including the number of peaks and average asymmetry index. We further tested our video module separately with and without using feature differences between consecutive frames. We compare the result of our audio module to that of sound wave classification with pattern recognition on spectrogram.

TABLE 3
Experimental results

| | Accuracy | Specificity | Sensitivity | AUC |
|---|---|---|---|---|
| Landmark + Asymmetry | 62.35% | 66.67% | 60.34% | 0.6350 |
| Video Module w/o | 70.28% | 11.11% | 98.24% | 0.5467 |
| difference | | | | |
| Video Module w/difference | 76.67% | 62.21% | 96.42% | 0.7281 |
| ResNet-18 | 58.20% | 42.75% | 70.22% | 0.5648 |
| VGG16 | 52.30% | 27.98% | 71.22% | 0.4960 |
| Audio Module Only | 70.24% | 40.74% | 84.21% | 0.6248 |
| Soundwave/Spectrogram | 68.67% | 59.26% | 77.58% | 0.6279 |
| Proposed Method | 79.27% | 66.07% | 93.12% | 0.7831 |
| Clinical Impression | 72.94% | 77.78% | 70.68% | 0.7423 |

As shown in Table 3 above, the proposed method outperforms all the strong baselines by achieving a 93.12% sensitivity and a 79.27% accuracy. The improvements of our proposed method from the baselines on accuracy are ranging from 10% to 30%. It is noticeable that proven image classification baselines (ResNet, VGG) are not ideal for our “in-the-wild” data. Comparing to the clinical impressions given by ER doctors, the proposed method achieves even higher accuracy and greatly improves the sensitivity, indicating that more stroke cases are correctly identified by our proposed approach. ER doctors tend to rely more on the speech abilities of the subjects and may overlook subtle facial motion weaknesses. Our objective is to identify real stroke and fake stroke cases among incoming subjects, who are already identified with high risk of stroke. If the patterns are subtle or challenging to observe by humans, ER doctors may have difficulty on those cases. We infer that the video module in our framework can detect those subtle facial motions that doctors can neglect and complement the diagnosis based on speech/audio. On the other hand, the ER doctors have access to emergency imaging reports and other information in the Electronic Health Records (EHR). With more information incorporated, we believe the performance of the framework can get further improved. It is also important to note that by using feature difference between consecutive frames for classification, the performance of the video module is greatly improved, validating our hypothesis about modeling based on motion.

Through the experiments, all the methods are experiencing low specificity (i.e., identifying non-stroke cases), which is reasonable because our subjects are subjects with suspicion of stroke rather than the general public. False negatives would be dangerous and could lead to hazardous outcome. We also took a closer look at the false negative and false positive cases. The false negatives are due to the labeling of cases using final diagnosis given based on diffusion-weighted MRI (DWI). DWI can detect very small lesions that may not cause noticeable abnormalities in facial motion or speech ability. Such cases coincide with the failures in clinical impression. The false positives typically result from background noise in audio, varying shapes of beard, or changing illumination conditions. A future direction is to improve specificity with more robust methods on both audio and video processing.

We also evaluate the efficiency of our approach. The recording runs for a minute, the extraction and upload of audio takes half a minute, the transcribing takes an extra minute, and the video processing is completed in two minutes. The prediction with the deep models can be achieved within seconds with GTX 1070. Therefore, the entire process takes no more than four minutes per case. If the approach is deployed onto a smartphone, we can rely on Google Speech-to-Text's real-time streaming method and perform the spatiotemporal frame proposal on the phone. Cloud computing can be leveraged to perform the prediction in no more than a minute, after the frames are uploaded. In such a case, the total time for one assessment should not exceed three minutes. This is ideal for performing stroke assessment in an emergency setting and the subjects can make self-assessments even before the ambulance arrives.

### CONCLUSIONS

We proposed a multi-modal deep learning framework for on-site clinical detection of stroke in an ER setting. Our framework is able to identify stroke based on abnormality in the subject's speech ability and facial muscular movements. We construct a deep neural network for classifying subject facial video, and fuse the network with a text classification model for speech ability assessment. Experimental studies demonstrate that the performance of the proposed approach is comparable to clinical impressions given by ER doctors, with a 93.12% sensitivity and a 79.27% accuracy. The approach is also efficient, taking less than four minutes for assessing one subject case. We expect that our proposed approach will be clinically relevant and can be deployed effectively on smartphones for fast and accurate assessment of stroke by ER doctors, at-risk subjects, or caregivers.

It should now be understood that the systems and methods described herein allow for on-site clinical detection of stroke in a clinical setting (e.g., an emergency room). The framework described herein can perform accurate and efficient stroke screening based on the abnormalities in the subject's speech ability and facial muscular movements. A dual branch deep neural network for the classification of subject facial video frame sequences and speech audio as spectrograms is used to capture subtle stroke patterns from both modalities. Experimental studies on the collected clinical dataset with real, diverse, “in-the-wild” ER subjects demonstrated that the proposed approach outperforms clinicians in the ER in providing a binary clinical impression on the existence of a stroke, with a 6.60% higher sensitivity rate and 4.62% higher accuracy when specificity is aligned. The framework has also been verified to be efficient and provide a screening result for the reference of clinicians in minutes.

While particular embodiments have been illustrated and described herein, it should be understood that various other changes and modifications may be made without departing from the spirit and scope of the claimed subject matter. Moreover, although various aspects of the claimed subject matter have been described herein, such aspects need not be utilized in combination. It is therefore intended that the appended claims cover all such changes and modifications that are within the scope of the claimed subject matter.

1. A method, comprising:
receiving, by a processing device, raw video of a subject presented for potential neurological condition;
splitting, by the processing device, the raw video into an image stream and an audio stream;
preprocessing the image stream into a spatiotemporal facial frame sequence proposal;
preprocessing the audio stream into a preprocessed audio component;
transmitting the facial frame sequence proposal and preprocessed audio component to a machine learning device that analyzes the facial frame sequence proposal and the preprocessed audio component according to a trained model to determine whether the subject is exhibiting signs of a neurological condition;
receiving, from the machine learning device, data corresponding to a confirmed indication of neurological condition; and
providing the confirmed indication of neurological condition to the subject and/or a clinician via a user interface.

2. The method of claim 1, further comprising transmitting additional information to the machine learning device, wherein the machine learning device uses the other information along with the facial frame sequence proposal and the preprocessed audio component to determine whether the subject is exhibiting signs of a neurological condition.

3. The method of claim 1, wherein the preprocessed audio component comprises one or more spectrograms, each of the one or more spectrograms representing an amplitude at each of a plurality of frequency levels over a period of time.

4. (canceled)

5. The method of claim 1, wherein the preprocessed audio component further comprises a speech transcription.

6. The method of claim 1, wherein receiving the raw video of the subject comprises receiving a video feed from a mobile device that has captured the subject repeating a predetermined sentence and describing a scene from a printed image.

7. The method of claim 1, wherein preprocessing the image stream into the spatiotemporal facial frame sequence proposal comprises extracting frontal face sequences from the raw video.

8. The method of claim 1, further comprising receiving a 3D depth data stream, wherein preprocessing the image stream comprises analyzing the 3D depth data stream to generate the spatiotemporal facial frame sequence proposal.

9. The method of claim 1, wherein preprocessing the image stream into the spatiotemporal facial frame sequence proposal comprises:
detecting a face of the subject with a face detection algorithm;
placing a rigid, square bounding box around the face of the subject;
tracking the face of the subject as the face moves;
estimating a pose of the face;
excluding any frame sequences outside one or more predetermined limits;
using a video stabilizer with a sliding window over a trajectory of between-frame affine transformations to smooth out pixel-level vibrations on one or more sequences; and
passing data corresponding to the one or more sequences are passed to an encoder.

10. The method of claim 1, wherein preprocessing the audio stream into the preprocessed audio component comprises:
using a video processing tool to extract the audio stream from the raw video and saving the audio stream as an audio file having a particular bit rate and frequency;
using an audio analysis software package to load the audio file and trim one or more silent edges;
performing a short-time Fourier transform on a sound waveform of the audio file and converting a scale of the audio file to decibel; converting a y-axis into a Mel-scale; and
saving a generated spectrogram therefrom.

11. The method of claim 1, wherein providing the confirmed indication of neurological condition further comprises providing supplemental information.

12. A system, comprising:
at least one processing device; and
a non-transitory, processor readable storage medium comprising programming instructions thereon that, when executed, cause the at least one processing device to:
receive raw video of a subject presented for potential neurological condition;
split the raw video into an image stream and an audio stream;
preprocess the image stream into a spatiotemporal facial frame sequence proposal;
preprocess the audio stream into a preprocessed audio component;
transmit the facial frame sequence proposal and preprocessed audio component to a machine learning device that analyzes the facial frame sequence proposal and the preprocessed audio component according to a trained model to determine whether the subject is exhibiting signs of a neurological condition;
receive, from the machine learning device, data corresponding to a confirmed indication of neurological condition; and
provide the confirmed indication of neurological condition to the subject and/or a clinician via a user interface.

13.-17. (canceled)

18. The system of claim 12, wherein the programming instructions that cause the at least one processing device to preprocess the image stream into the spatiotemporal facial frame sequence proposal comprises programming instructions for:
detecting a face of the subject with a face detection algorithm;
placing a rigid, square bounding box around the face of the subject;
tracking the face of the subject as the face moves;
estimating a pose of the face;
excluding any frame sequences outside one or more predetermined limits;
using a video stabilizer with a sliding window over a trajectory of between-frame affine transformations to smooth out pixel-level vibrations on one or more sequences; and
passing data corresponding to the one or more sequences are passed to an encoder.

19. The system of claim 12, wherein the programming instructions that cause the at least one processing device to preprocess the audio stream into the preprocessed audio component comprises programming instructions for:
using a video processing tool to extract the audio stream from the raw video and saving the audio stream as an audio file having a particular bit rate and frequency;
using an audio analysis software package to load the audio file and trim one or more silent edges;
performing a short-time Fourier transform on a sound waveform of the audio file and converting a scale of the audio file to decibel; converting a y-axis into a Mel-scale; and
saving a generated spectrogram therefrom.

20. (canceled)

21. A non-transitory storage medium, comprising programming instructions thereon for causing at least one processing device to:
receive raw video of a subject presented for potential neurological condition;
split the raw video into an image stream and an audio stream;
preprocess the image stream into a spatiotemporal facial frame sequence proposal;
preprocess the audio stream into a preprocessed audio component;
transmit the facial frame sequence proposal and preprocessed audio component to a machine learning device that analyzes the facial frame sequence proposal and the preprocessed audio component according to a trained model to determine whether the subject is exhibiting signs of a neurological condition;
receive, from the machine learning device, data corresponding to a confirmed indication of neurological condition; and
provide the confirmed indication of neurological condition to the subject and/or a clinician via a user interface.

22. The non-transitory storage medium of claim 21, wherein the preprocessed audio component comprises one or more spectrograms, each of the one or more spectrograms representing an amplitude at each of a plurality of frequency levels over a period of time.

23. (canceled)

24. (canceled)

25. The non-transitory storage medium of claim 21, wherein the programming instructions for causing the at least one processing device to receive the raw video of the subject comprises programming instructions for receiving a video feed from a mobile device that has captured the subject repeating a predetermined sentence and describing a scene from a printed image.

26. The non-transitory storage medium of claim 21, wherein the programming instructions for causing the at least one processing device to preprocess the image stream into the spatiotemporal facial frame sequence proposal comprises programming instructions for extracting frontal face sequences from the raw video.

27. The non-transitory storage medium of claim 21, wherein the programming instructions for causing the at least one processing device to preprocess the image stream into the spatiotemporal facial frame sequence proposal comprises programming instructions for:
detecting a face of the subject with a face detection algorithm;
placing a rigid, square bounding box around the face of the subject;
tracking the face of the subject as the face moves;
estimating a pose of the face;
excluding any frame sequences outside one or more predetermined limits;
using a video stabilizer with a sliding window over a trajectory of between-frame affine transformations to smooth out pixel-level vibrations on one or more sequences; and
passing data corresponding to the one or more sequences are passed to an encoder.

28. The non-transitory storage medium of claim 21, wherein the programming instructions for causing the at least one processing device to preprocess the audio stream into the preprocessed audio component comprises programming instructions for:
using a video processing tool to extract the audio stream from the raw video and saving the audio stream as an audio file having a particular bit rate and frequency;
using an audio analysis software package to load the audio file and trim one or more silent edges;
performing a short-time Fourier transform on a sound waveform of the audio file and converting a scale of the audio file to decibel; converting a y-axis into a Mel-scale; and
saving a generated spectrogram therefrom.

29. The non-transitory storage medium of claim 21, wherein the programming instructions for providing the confirmed indication of neurological condition further comprises programming instructions for providing supplemental information.

30.-31. (canceled)
